(19) United States
(12) Patent Application Publication
(10) Pub. No.: US 2019/0134417 A1
(43) Pub. Date: May 9, 2019

(54) PORTABLE REPETITIVE TRANSCRANIAL MAGNETIC STIMULATION APPARATUS

(71) Applicant: Han TONG, New Taipei (TW)

(72) Inventor: Han TONG, New Taipei (TW)

(21) Appl. No.: 16/182,820

(22) Filed: Nov. 7, 2018

(30) Foreign Application Priority Data
Nov. 9, 2017 (TW) 106216677
Feb. 14, 2018 (TW) 107105606

(51) Int. Cl. A61N 2/02; H01F 27/28; H01F 27/245; H01F 27/02; H02M 3/07; H02M 1/34

(52) U.S. Cl. CPC A61N 2/02; H01F 27/28; H01F 27/245; H02M 2001/342; H02M 3/07; H02M 1/34; H02M 2001/348; H01F 27/02

(57) ABSTRACT

A portable repetitive transcranial magnetic stimulation (rTMS) apparatus is provided. The rTMS apparatus includes an upper fastening component, a lower fastening component, a driver circuit and an inductor which is electrically connected to the driver circuit and used as a stimulator. The inductor is formed of a core and a coil. The core has a groove. The coil includes an upper part and a lower part. The upper part of the coil is configured to be distal to the core and pass through an upper side, a left side or a right side of the core. The lower part of the coil is configured to pass through the groove.

### FIELD OF THE INVENTION

The present invention relates to a portable repetitive transcranial magnetic stimulation (rTMS) apparatus. Specifically, the portable rTMS apparatus of the present invention controls operations of driver circuit loop respectively via pulse generators, and a specific designed stimulator is used in combination with the portable rTMS apparatus to achieve the purpose of being portable.

### DESCRIPTION OF THE RELATED ART

A common repetitive transcranial magnetic stimulation (rTMS) apparatus currently available has a relatively large volume and must be connected to the wall outlet when using, so the use thereof is limited by location and in most cases, a patient can only receive the rTMS treatment at the location where the rTMS apparatus is located. Moreover, the stimulator of the rTMS apparatus will generate noise during the operation, and the temperature of the stimulator also increases gradually as the operating time increases so that the stimulator cannot be used if the temperature thereof is too high.

Accordingly, an urgent need exists in the art to provide a portable rTMS apparatus which can be used, while not limited by location.

### BRIEF SUMMARY OF THE INVENTION

An objective of the present invention is to provide a portable repetitive transcranial magnetic stimulation (rTMS) apparatus, cooperate with the newly designed stimulator (an inductor) and circuit to remarkably reduce the overall volume of the apparatus, and further reduce the power consumption and achieving the feature of being portable. Accordingly, as compared to the conventional rTMS apparatus, the portable rTMS apparatus of the present invention is not limited by location when using, and can be powered by a built-in battery or a mobile power supply.

To achieve the aforesaid objective, the present invention discloses a portable rTMS apparatus, which comprises a driver circuit and an inductor. The inductor is electrically connected to the driver circuit and is used as a stimulator. The inductor includes a core and at least one coil. The core has a groove. The at least one coil has an upper part and a lower part. The upper part of the at least one coil is configured to be distant to the core and pass through the upper side, the left side or the right side of the core. The lower part of the at least one coil is configured to pass through the groove of the core.

After reading the preferred embodiments and the appended drawings, people skilled in this field may understand the technical features, implementation or other purpose of the claimed invention.

### BRIEF DESCRIPTION OF THE DRAWINGS

FIG. 1 is a schematic view depicting the appearance of a portable repetitive transcranial magnetic stimulation (rTMS) apparatus 1 within the present invention;

FIG. 2A is a schematic view depicting a cross-sectional structure of a core COE;

FIG. 2B is a schematic view depicting a structure of the core COE;

FIG. 2C to FIG. 2F are schematic views depicting the cross-sectional view of the core COE and the arrangement of a lower part of the coil;

FIG. 2G to FIG. 2H are schematic views depicting the bending and extending of the core COE;

FIG. 2I is a schematic view depicting the core COE;

FIG. 3A is a view depicting the magnetic flux distribution of the core COE;

FIG. 3B to FIG. 3D depict the arrangement of an upper part of a coil COL and a magnetic field distribution simulated diagram of the core COE;

FIG. 3E to FIG. 3F depict the shape of the upper part of the coil COL and the magnetic field distribution simulated diagram of the core COE;

FIG. 4A to FIG. 4C are schematic views depicting the arrangement of the upper part of the coil COL;

FIG. 5A to FIG. 5C are schematic architectural views depicting an upper fastening component UFC and a lower fastening component LFC;

FIG. 6A is a schematic structural view depicting a ceramic substrate;

FIG. 6B is a schematic view depicting the arrangement of ceramic substrates;

FIG. 7A to FIG. 7C are schematic views depicting structural relationships between ceramic substrates and extended ceramic substrates;

FIG. 8A is a schematic view depicting the arrangement of an upper blocking component;

FIG. 8B is a schematic view depicting the arrangement of a lower blocking component and a magnetostrictive material;

FIG. 9A to FIG. 9E are schematic views depicting the deformation of the core COE;

FIG. 10 is a schematic view depicting an inductor disposed within a housing;

FIG. 11A and FIG. 11B are schematic views depicting the circuit of the portable rTMS apparatus 1 within the present invention;

FIG. 12A depicts the changing current of inductor L when charging and discharging;

FIG. 12B depicts the current direction of the inductor L within a time T1;

FIG. 12C depicts the current direction of the inductor L within a time T2;

FIG. 12D depicts the current direction of the inductor L within a time T3;

FIG. 12E depicts the current direction of the inductor L within a time T4;

FIG. 13 to FIG. 14 depict schematic views of circuits of the portable rTMS apparatus 1 for different embodiments within the present invention;

FIG. 15A to FIG. 15C are schematic views depicting the passive snubber SB;

FIG. 16A to FIG. 16C, FIG. 17A to FIG. 17B and FIG. 18 to FIG. 35 are schematic views depicting the circuit of the portable rTMS apparatuses 1 according to different embodiments within the present invention;

FIG. 36A is a schematic view depicting circuits connected in parallel of the portable rTMS apparatus 1 within the present invention;

FIG. 36B is a schematic view depicting circuits connected in series of the portable rTMS apparatus 1 within the present invention;

FIG. 36C is a schematic view depicting circuits connected in series and in parallel of the portable rTMS apparatus 1 within the present invention; and

FIG. 37 is a schematic view depicting a fastening device within the present invention.

### DESCRIPTION OF EMBODIMENTS

In the following description, the present invention will be explained with reference to embodiments thereof. It shall be appreciated that, these embodiments within the present invention are not intended to limit the present invention to any particular environment, applications or special mode described in these embodiments. Therefore, description of these embodiments is only for purpose of illustration rather than to limit the present invention, and the scope claimed in this application shall be governed by the claims. Besides, in the following embodiments and the attached drawings, elements unrelated to the present invention are omitted and not depicted; and dimensional relationships among individual elements in the attached drawings are illustrated only for ease of understanding, but not to limit the actual scale. Unless otherwise defined, material properties are all measured at a room temperature.

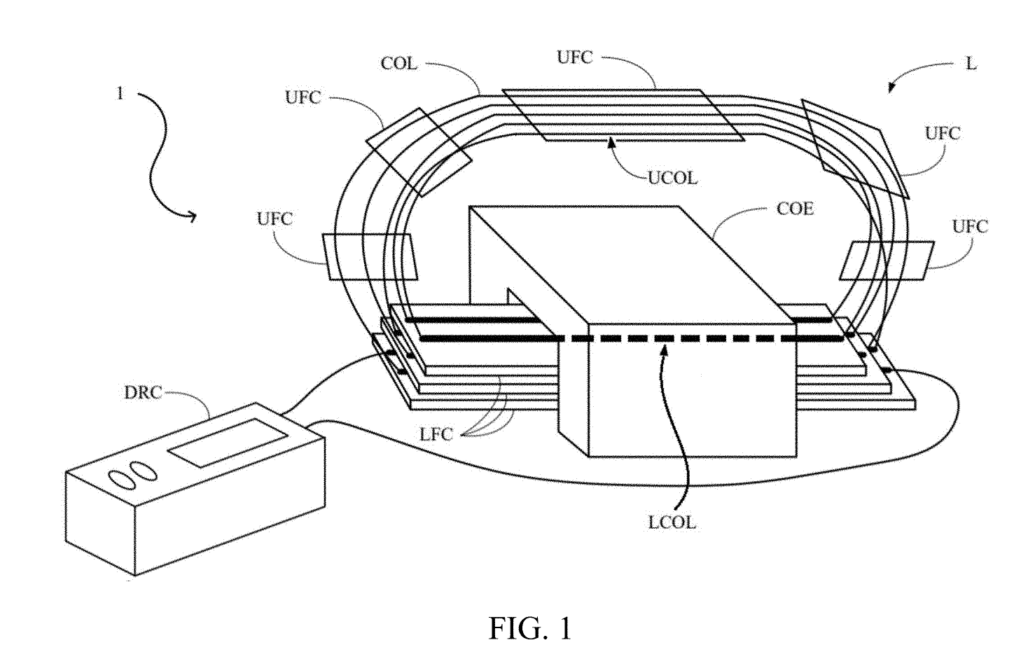

A first embodiment of the present invention is as shown in FIG. 1 to FIG. 10. FIG. 1 is a schematic view depicting the appearance of a portable repetitive transcranial magnetic stimulation (rTMS) apparatus 1 within the present invention. The portable rTMS apparatus comprises an upper fastening component UFC, a lower fastening component LFC, a driver circuit DRC and an inductor L. The driver circuit DRC may be encapsulated within a housing, and the inductor L may be disposed within another housing (not shown). A controller, a screen, a button and a switch may be additionally provided on the housing of the driver circuit DRC. The portable rTMS apparatus may be turned on, turned off or perform other operations via the controller. The controller may respond to actions of the user, such as operating the button, operating the switch, touching the screen or other related operation.

The inductor L is electrically connected to the driver circuit DRC and is used as a stimulator. The inductor L includes a core COE and at least one coil COL. The core COE has a groove CG, a coil COL has an upper part UCOL, a lower part LCOL and two connecting portions INT. The upper part UCOL of the coil COL is configured to be distant to the core COE and pass through an upper side, a left side or a right side of the core COE, and is fastened by the upper fastening component UFC. The lower part LCOL of the coil COL is configured to pass through the groove CG and is fastened by the lower fastening component LFC.

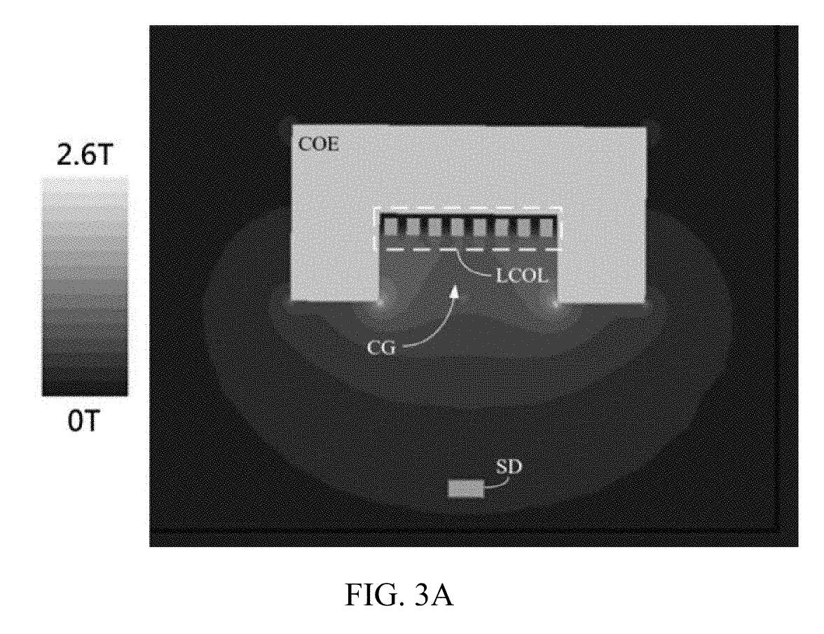

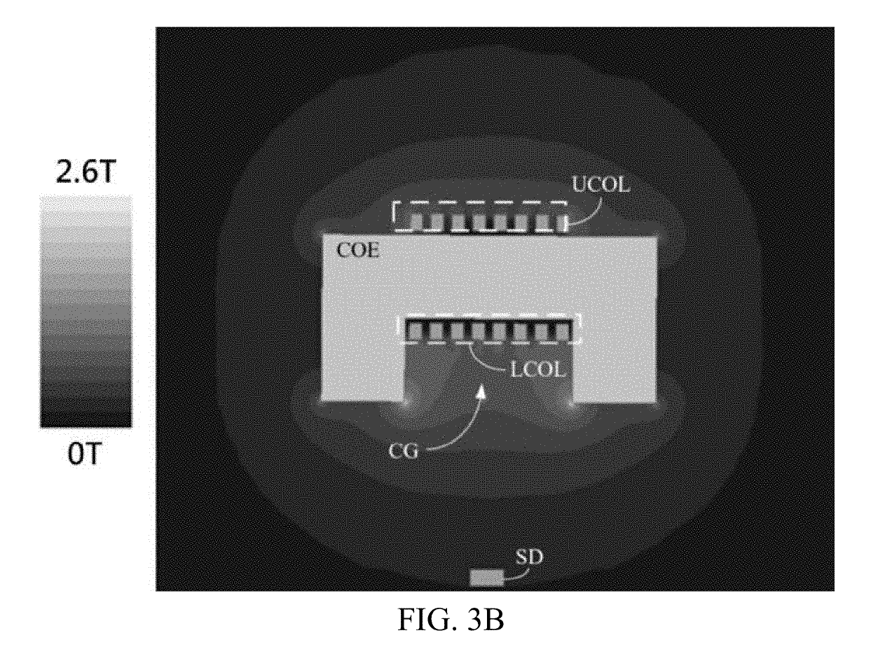

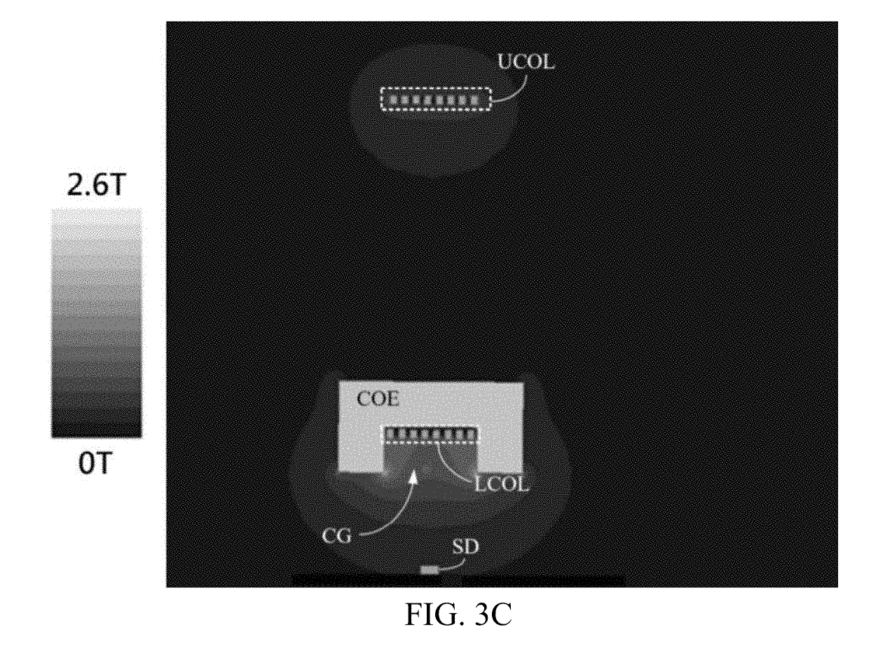

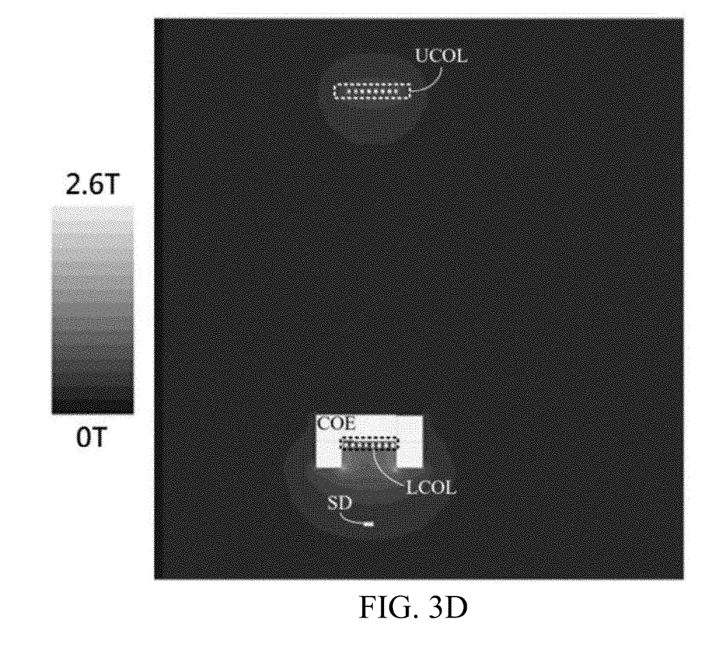

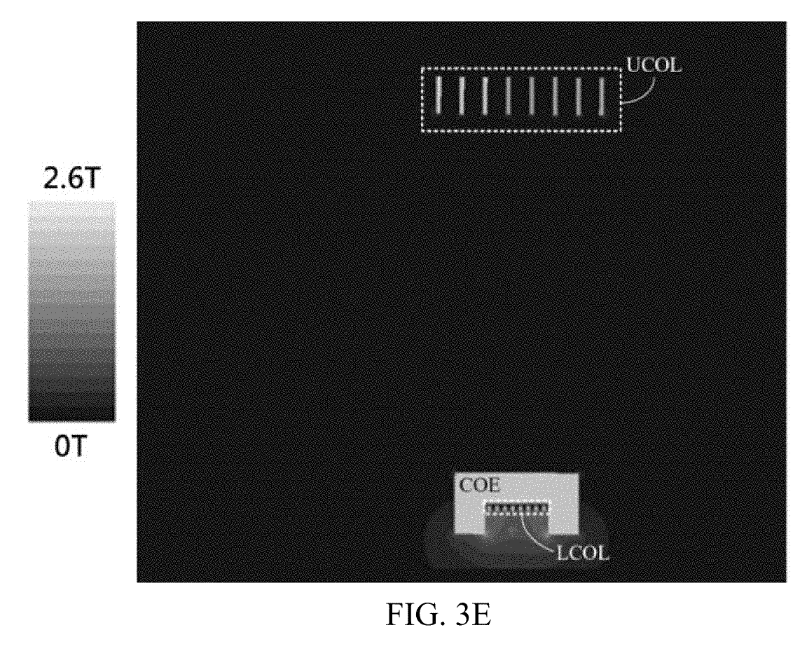

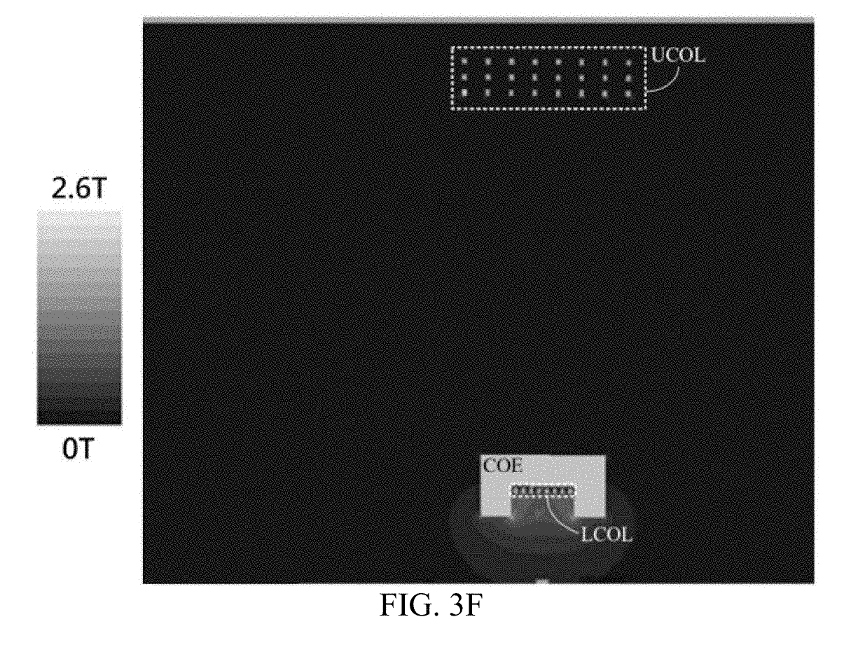

Concretely speaking, as shown in FIG. 3B to FIG. 3F, the coil COL that is located within some two section plane of the core COE are the upper part UCOL and the lower part LCOL, respectively. The lower part LCOL is the stimulating portion of the stimulator, and the upper part UCOL of the coil COL is responsible for flowing back the current of the lower part LCOL. Therefore, the current direction of the upper part UCOL is opposite to the current direction of the lower part LCOL. For example, as seen from the section plane of the core of FIG. 3C, if the direction of the lower part LCOL is in-to-plane, then the direction of the upper part UCOL is out-of-plane. In addition, conductors outside the some two section plane of the core COE (i.e., conductors of the connecting portion INT, which do not belong to the conductors of the upper part UCOL and the lower part LCOL) belong to the transitional conductors. A single coil should comprise at least two connection terminals. If two coils are winded on the core COE, and four connection terminals of the two coils are not connected with each other, then the two coils can be regarded as two inductors with mutual coupling, as shown in FIG. 23, FIG. 24 and FIG. 25. If at least two coils are winded on the core COE, and two terminals of different coils are connected, then the coils can be regarded as a center-tapped inductor with three connection terminals, as shown in FIG. 26, FIG. 27, FIG. 28, FIG. 29, FIG. 30 and FIG. 31.

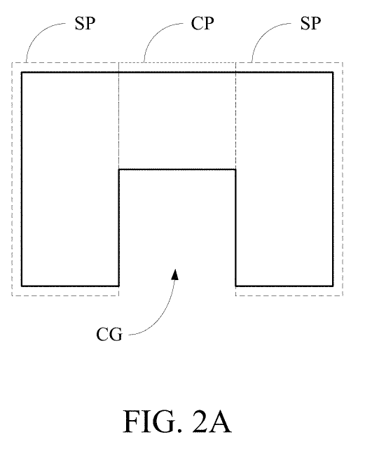

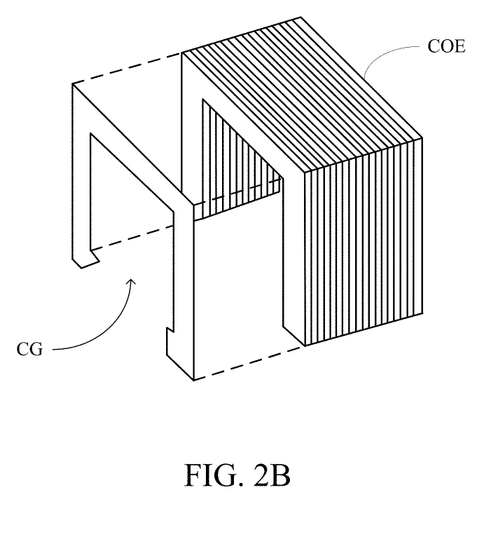

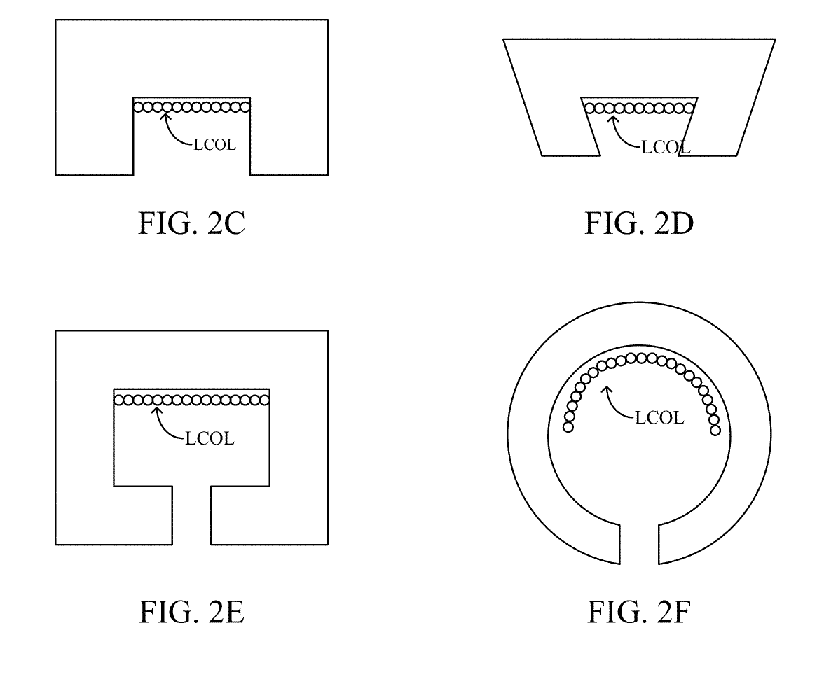

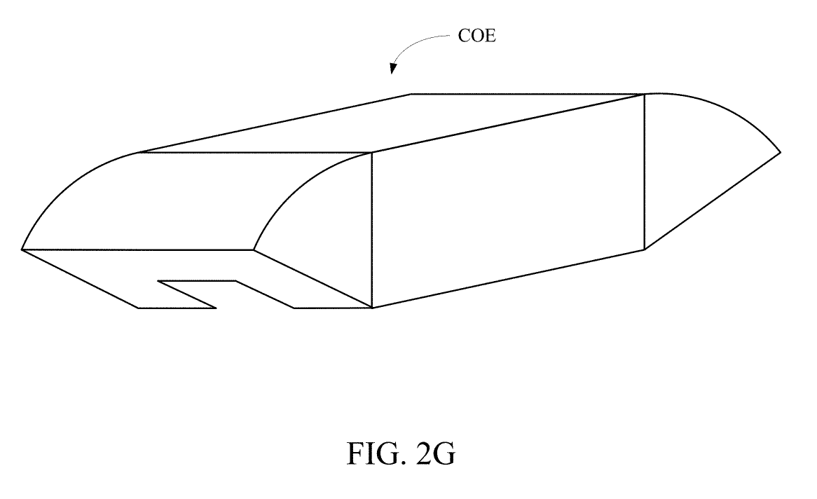

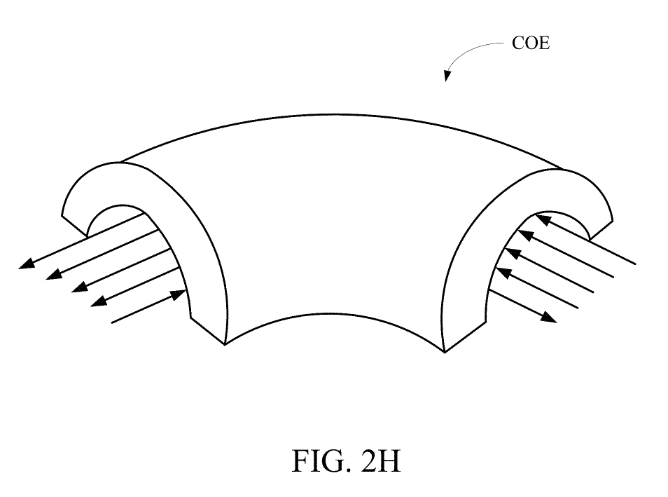

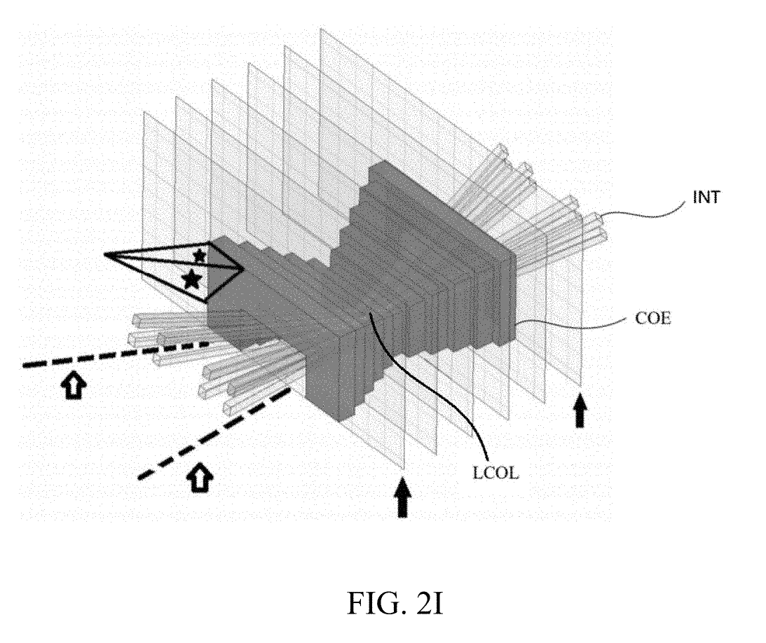

FIG. 2A is a schematic view depicting a cross-sectional structure of a core COE. The core COE has two side portions SP and a connecting portion CP. The core COE may be a ferromagnetic material, a ferrimagnetic soft magnetic material or a high saturation flux density material (e.g., an alloy containing Fe and Co) with a saturation density greater than 0.5 Tesla (T). When comparing a low saturation flux density material that is saturated to a high saturation flux density material that is under nearly-saturation operation, if the ratio of the saturation flux densities of the two materials is 3:2, then the magnetic resistance and the current required for generating the same magnitude magnetic field will be reduced by about 75%. The conductors of the coil COL may be copper wires, aluminum wires or other conductors (e.g., silver). The conductors of the coil COL pass through the groove CG of the magnetic core COE via the groove CG.

The common types of the core COE are: silicon steel sheet, ferrocobalt alloy, ferronickel alloy, magnetic powder core, amorphous, nanocrystalline, and so on. All of they usually have saturation densities greater than 0.5 T. The saturation densities of the silicon steel sheet and the ferronickel alloy are greater than 1.3 T, wherein the saturation density of the ferrocobalt alloy is even greater than 1.9 T. Although the aforesaid three alloys have good saturation density properties, the internal impedance thereof is relatively poor. Therefore, in order to enhance the impedance to resist the eddy current, the core COE is often structurally designed as sheets which are substantially perpendicular to the current direction, as shown in FIG. 2B. For example, the core COE may be formed of a plurality of iron core sheets. When the core COE is made of a high saturation flux density material, and the saturation density of the core COE is greater than 1.3 T, the thickness of each iron core sheet may be smaller than 1 mm. Besides, when the saturation density of the core COE is greater than 1.9 T, the thickness of each iron core sheet may be smaller than 0.5 mm. Furthermore, an insulating layer is presented between the sheets (i.e., between iron core sheets) to reduce the eddy current within the core COE.

Moreover, in other embodiments, the core COE may also be made of different materials (e.g., a magnetic powder core, an amorphous and a nanocrystalline) so that the core COE has built in an internal high-impedance structure, thereby reducing the eddy current within the core COE without using the sheet-shaped structure. Because there is a large airgap in the stimulator, so an airgap in the core is also allowable, or the core can be formed by a combination of a plurality of small cores without influencing the operation. The core saturation test adopts the Epstein frame measurement, and a magnetic field intensity of 1200 A/m is selected.

The core COE may have various extended shapes, and the cross sections thereof are as shown in FIG. 2C to FIG. 2F. A conductor layer made of conductors with the same orientation at the lower part of the coil COL passes through the groove CG. When comparing to a plate-shaped core (as known as a groove with depth equal to 0 mm), the grooved core (as known as an inverted U-shaped core) has relevance with the direct current (DC) loss, alternating current (AC) loss and thermal damage risk to the user. For DC losses: In a plate-shaped core, if a lower DC loss is desired, the thickness of the conductor layer would need to be increased in the plate-shaped core. Then, the distance between the core and the average current must increase. The-conductor would gradually lose its advantages provided by core, and become a conductor with air core property. Therefore, either a thicker conductor or a conductor closer to the core can solely be chosen. For a grooved core, due-to the two side extended portions SP, a shorter distance between core and conductor can still be provided while increasing the thickness of the conductor, and the magnetic field generated is still superposed at the lower part. Therefore, both benefits can be acquired at the same time.

Considering AC losses, there must be a fixed magnitude in the magnetic field for stimulation to be effective for the user. In the case where the width is much larger than the thickness in a conductor layer, because magnetic field only distributes downward, there is no great difference between the effects provided by the plate-shaped core or the groove core. For example, considering a conductor layer with a width of 3 cm and a thickness of 0.1 cm, providing a groove of 12 mm only reduces the loss by 20% at the frequency of 4 kHz. However, due to the correlation with DC losses, the conductor layer cannot be made too thin, otherwise, the DC loss will be excessively large. Furthermore, for a thicker conductor layer, the magnetic field of the plate-shaped core would not only distribute downward, but also distribute leftward and rightward. Unlike the plate-shaped core, the magnetic field of the groove core would still distribute only downward due to the two side extended portions SP. Without the left and right portions exposed under the magnetic field, the proximity effect can be effectively reduced in the conductor layer, hence the AC loss is also reduced. For example, comparing to the plate-shaped core (as known as a groove with depth equal to 0 mm), the power consumption is reduced by more than 45% at the frequency of 4 kHz in a conductor layer with a width of 3 cm, a thickness of 6 mm, and a groove depth of 12 mm.

For thermal damage consideration: When the grooved core has two side portions SP extended beyond the lowest part of the conductor layer, because these distal ends of the two side portions SP serve as the proximate part of the stimulator and are-close to human body, such a configuration would make the conductor farther away from the human body, and the thermal damage to the human body by the coil COL is therefore reduced. Besides, the reduction of DC loss and AC loss will also reduce heat generation on their account.

The dimensional design of a core COE may enable the conductor to be at a distance from distal ends of the two side portions SP (e.g., the depth of the groove CG is 12 mm, the thickness of the conductor layer is 6 mm), so the thickness of the conductor layer is less than the depth. At least, the depth of the groove should cover the position of the average current of the lower conductor. For example, for a conductor layer of 6 mm, because the average current is at 3 mm, the depth of the groove should at least be larger than 3 mm in order to provide an obvious effect. The function gradually improves while the depth of the groove increases, and the function improvement is very obvious when the depth is about a half of the width of the opening of the core. When the depth of the groove is about half to one times the width of the groove, the function improves as the depth of the groove increases, but it is not obvious. When the depth of the groove is beyond one times the width of the groove, the function is less likely to improve as the depth of the groove increases. The shape of the core may change, so there is no absolute range theoretically. However, one suitable range of the depth of the groove that found in experiment is from 0.7 cm to 4 cm when considering weight and power consumption for portability.

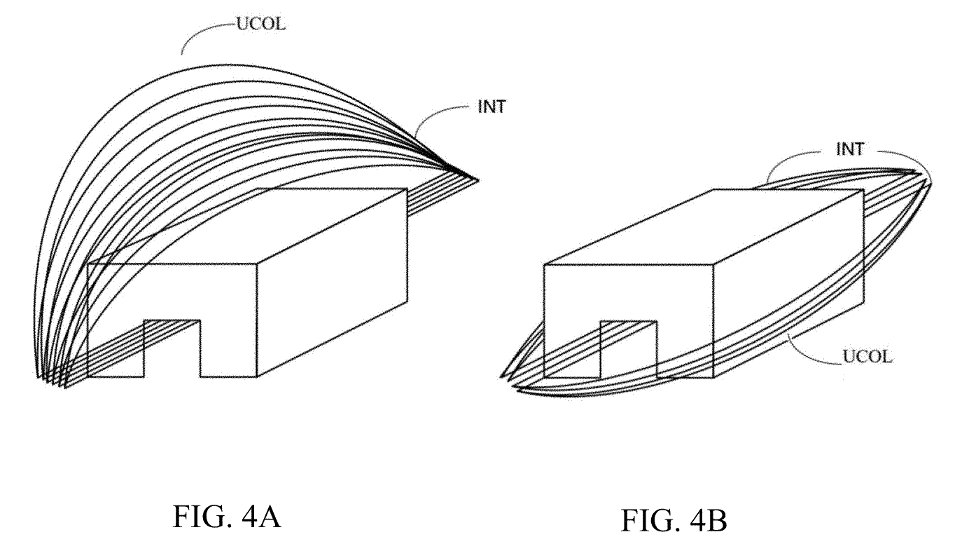

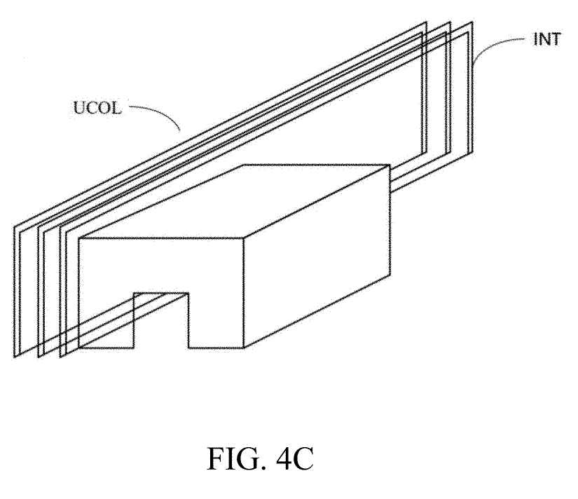

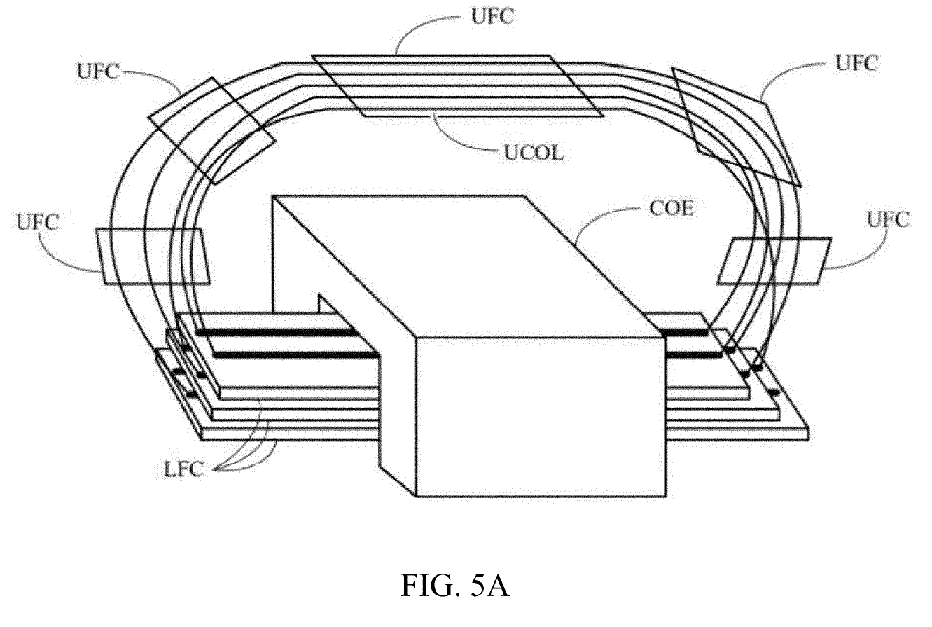

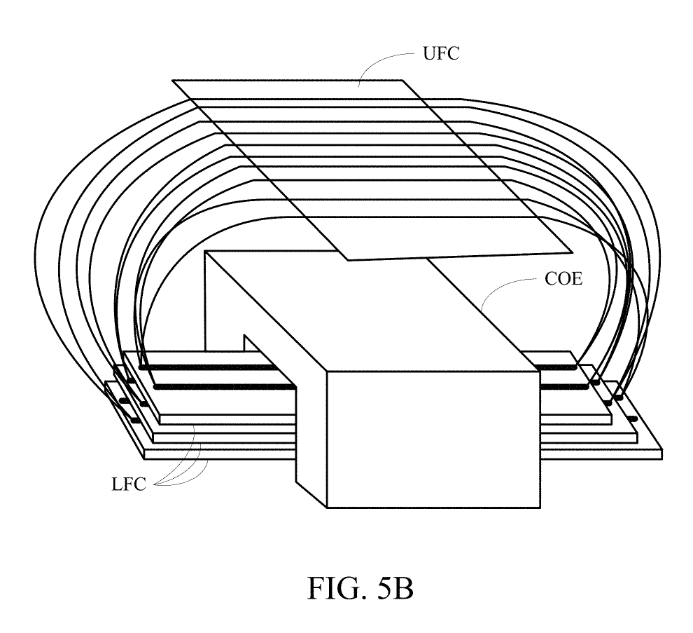

As compared to the lower part LCOL, the distance is greater between the average current of the upper part UCOL of the coil COL and the core COE, and the ratio is in the extent of more than two to one. The distance between the average current of the upper part UCOL of the coil COL and the core COE and the distance between the average current of the lower part LCOL of the coil COL and the core COE are calculated in the following way: (1) firstly, taking an current in space average vector of a single conductor; (2) then, taking an absolute value of the distance between the vector in space and the main body of the core; and (3) taking an average of the absolute values of the distances of all the conductors. The reason of using such a definition is as shown in FIG. 4B, for the upper part UCOL which is away from the core COE and has a suitable structure, the direct sum of vectors in space of all the conductors would fall in the core COE, and thus causes misjudgment. Adopting the aforesaid definition of (1) to (3), the distance can then comply with intuitive explanation that the upper part UCOL is farther away from the core COE.

For the upper coil in a grooved core, the upper part of the coil that is away from the core has some relevance-with the DC loss, the AC loss, the weight of the capacitor and the weight of the core. In the case of a conventional wound-type core, only one of the power consumption or the weight of the core can be prioritized. For example, if an upper coil of a larger cross-sectional area is adopted for better DC loss, the size of the groove core needs to be increased accordingly (because the cross-sectional area of the lower coil is the same as that of the upper coil, and the current of the lower coil needs to pass through the groove of the core), and the weight thereof also increases. When the upper coil is away from the core, the weight and the power consumption can be chosen at the same time (for the DC part, the cross-sectional area of the lower coil and the upper coil may be different, and the AC part is related to distribution in space). For example, in the case where the core and the lower coil does not change so that the function is not affected, adopting an upper coil of a larger cross-sectional area can reduce the DC loss and the AC loss, and adopting a plate-shaped, distributed upper coil can reduce the AC loss. Moreover, by keeping the upper part of the coil away from the core, the magnetic flux and inductance generated by the upper part of the coil with the core can be reduced. Saturation of the core can be prevented by reducing the magnetic flux generated by the core with the upper part of the coil, thereby reducing the thickness and weight of the core. The weight of the capacitor in the driver loop can be lowered by reducing the inductance generated by the core with the upper part of the coil. The weight of the capacitor and the weight of the core are the most significant parts among the components that are essential to the whole device.

The width of the connecting portion CP of the groove CG, the left-to-right width of the groove CG, including the lower core opening width and the maximum width where the conductor passes through, when taking size and weight factors into consideration, should all be in the extent of from 0.7 cm to 11.2 cm in order to coordinate with the stimulating depth. In order to prevent the saturation of the core, the thickness of the core COE should be in the extent from 0.4 cm and 4 cm. When the width of the opening of the core is further designed to be in the extent from 1.4 cm to 5.6 cm, and the thickness of the core COE is further designed to be in the extent from 0.7 cm to 2.8 cm, a better stimulating strength per unit weight can then be achieved. The lower part LCOL of the coil COL should be arranged from the position close to the inner edge of the groove CG, as shown in FIG. 2C to FIG. 2F; and if more coils are required, then the coils are superimposed downward. The width of the opening, the length and the height of the whole core should all be smaller than 11.2 cm to maintain the size and weight for portablity.

The leftward, rightward, downward or upward bending angles of the average current of the lower part LCOL of the coil COL at the entry and exit direction need to be within 60 degrees. For example, in FIG. 2H, the bending angle should be within 60 degrees when bending to the left. As shall be appreciated by those with ordinary skill of the art, according to the Biot-Savart law, the final magnetic field is determined by the integral sum of the current and the permeability of materials. The core COE may also bend and extend downward along the front end and the back end as shown in FIG. 2G, or bend toward one of the SP sides as shown in FIG. 2H. However, the difference of the magnetic field will not be too large according to the Biot-Savart law. Similarly, in FIG. 2H, the arrow indicates the current direction: the current direction on most of the conductors are the same, while the current direction in one conductor is opposite to the current direction of other conductors. According to the Biot-Savart law, the difference of the magnetic field caused by it will not be too large. Among the conductors of the lower part LCOL of the coil COL, the conductors with the current direction angle within 60° may be regarded as having the same direction, and they should represent more than 80% of all the conductors.

The required function of the stimulator can be achieved simply by some rectangular structures. However, in case if a core with an inordinate shape needs to be adopted, reference is made to FIG. 2I for the design principle. The rTMS apparatus will surely have a groove CG of the-grooved core COE, and a lower part LCOL of the coil COL that passes through the groove CG, and they should be closer to the user site than the connecting portion CP of the core COE, and the upper part UCOL of the coil COL. An average current direction is taken by the lower part LCOL of the coil COL with the part passing through the groove CG (i.e., the average current should pass through the groove of the core in the space), the average current direction is the normal vector of the tangential plane, and an entry tangential plane and an exit tangential plane can be defined for the core COE, as shown by the black arrow of FIG. 2I. The tangential planes are selected based on the principle of defining a core COE which is a bit shorter, while giving up the pieces that stand out from the rest, such as the star part of FIG. 2I. The reason to give up the pieces that stand out from the rest is that these pieces are incapable of-providing an improved function for the apparatus. The entry tangential plane and the exit tangential plane should be capable of defining the lower part LCOL of the coil COL that is initially selected, e.g., the part of eight conductors that is located between the two tangential planes shown in FIG. 2I. If there is a great difference, then the aforesaid steps are repeated and iterated to obtain the ideal tangential planes. The tangential plane is important because it can provide the following functions.

Firstly, it provides a judgement on the parameters mentioned in the above paragraphs: the width of the groove, the depth of the groove, the thickness of the core, the range of the upper part of the coil, the distance between the upper part of the coil and the core, and the distance between the lower part of the coil and the core. In case the stimulator is divided uniformly among these tangential planes between the entry tangential plane and the exit tangential plane, at least a 80% of sections should correspond to the parameters provided in the above paragraphs, while exact correspondence is not strictly required for every section, but the breadth should not deviate more than itself for at least 80% in these sections, and the core is conformed to a groove-shaped one.

Secondly, it can be used to determine the bending angle of the lower part of the coil: when the middle section between the entry tangential plane and the tangential plane going inward by 40% is defined as the entrance lower coil, and the middle section between the exit tangential plane and the tangential plane going inward by 40% is defined as the exit lower coil, then the average current of the entrance lower coil and that of the exit lower coil can be calculated respectively, from which the deviation angle can be calculated. Furthermore, in an inordinate core, because the lower coil should still be close to the core, the direction of different conductors within the lower part of the entrance coil may be different, as shown by the white arrow of FIG. 2I. However, 80% of the conductors should still have mutual offset angles less than 60 degrees, as it may be imagined that 80% of the conductors have directions in a cone with 60-degree pointing angle.

Thirdly, the two tangential planes may be used to distinguish: the upper part of the coil; the lower part of the coil, which is between the two tangent planes; and the part excluded, which can be regarded as interconnect part coil. The distance between the connecting coil and the core is not particularly limited and the connecting coil has a transitional property. The property of the part of the connecting coil that is close to the lower coil may not be similar to that of the lower coil because the distance between the part and the core is not as close as the distance between the lower coil and the core. However, the property of the part of the connecting coil that is close to the upper coil is similar to that of the upper coil because the distance between the part and the core is as far as the distance between the upper coil and the core. Fourthly, it is used to determine the length of the core, and the length of the core should range from 0.7 cm to 11.2 cm to maintain the portability. If the length is further designed to be between 1.4 cm and 5.6 cm, then a better stimulating strength can be achieved upon each unit energy.

The width of the groove CG of the core COE needs to be selected according to the depth to be stimulated by the rTMS apparatus. For example, if the stimulating site is at the depth of 2.5 cm, as shown by a stimulating site SD of FIG. 3A, then the width of the lower opening of the groove CG shall be selected to be about 2.8 cm. Furthermore, the magnetic field of a conventional coil can distribute in six directions in an open space, i.e., upward, downward, leftward, rightward, frontward, and backward. If the design is modified into FIG. 3A, then only the downward distribution is left, i.e., only about one quarter of the energy is needed for magnetic field generation. Such a width selection may further create sufficient magnetic field gradient, and finally, it is the magnetic field gradient, rather than the absolute intensity, which is used to generate the stimulating electrical field. As a result, selecting the bent distributed magnetic field intensity will be more effective than selecting the straight distributed magnetic field intensity. The difference between some prior arts and this invention lies in that: the length of core groove and the conductor in some prior arts are greater than 11.2 cm, and it would make the refluxing current more diversified in the stimulating part of the brain. However, the portable rTMS apparatus 1 selects the core groove and the conductor with lengths ranges from 0.7 cm to 11.2 cm, and it become not sure that the current will be more diversified in the brain. The objective of some prior arts is to provide a larger absolute magnetic field intensity at a depth (e.g., of 40 mm) within the brain so that a larger stimulating part and refluxing part are generated by the core, the portable rTMS apparatus 1 of the present invention, on the other hand, does not emphasize to increase the absolute magnetic field strength at a depth within the brain, but rather, it's main design emphasis is on “reducing the power consumption” and “reducing the weight”.

Moreover, in FIG. 3A, if the stimulating site SD is at a depth of 2.5 cm, then a width of 2.8 cm of the groove CG may make the bending part located near the stimulating position. If an excessively large width (e.g., a conventional circular coil larger than 11.2 cm) is used, and the absolute intensity of the magnetic field is increased while the gradient is not increased, the design is therefore not being advantageous for the stimulating site SD. Moreover, the thickness of the core COE is selected mainly for preventing the saturation, and in practice, the thickness is greater than about 0.4 cm and smaller than about 4 cm. The length may also be designed based on the gradient principle, e.g., when the width of the lower opening of the groove CG is 2.8 cm, the length can also designed to be 2.8 cm, so that the gradient generated by the width and the length can be similar.

Distances between the upper part UCOL of the coil COL and the core COE are 0 cm, 6 cm and 12 cm in FIG. 3B to FIG. 3D respectively. Taking a model having a length of 5 cm in the direction normal to the plane, a frequency of 10 kHz and a power-on time of 0.2% as an example, if the way of winding of FIG. 3B is used to generate a magnetic field with a maximum value of 0.2 T at the depth of 2 cm, it can be calculated that the maximum energy storage in the total inductor is 14 J, and the AC loss is 8.14 W. If the upper part UCOL of the coil COL is moved farther away from the core by 6 cm, as shown in FIG. 3C, then the maximum energy storage is changed to 12 J, and the power consumption is changed to 5.44 W. In other words, as the upper part UCOL of the coil COL is moving away from the core COE, then under the same stimulating strength, the total energy storage can be changed to 86%, and the power consumption can be changed to 70%. If the distance between the upper part UCOL of the coil COL and the core COE is further increased to 12 cm, as shown in FIG. 3D, it can be observed that the power consumption and the energy storage are not changed anymore.

Other than keeping the upper part UCOL of the coil COL and the core COE away, the shape of the upper part UCOL of the coil COL also influences the power consumption and the energy storage. As shown in FIG. 3E, the upper part UCOL of the coil COL is formed of eight conductor sheets having a total width of 5.4 cm and a total height of 1.2 cm, and thus the total energy storage can be changed to 11 J, and the total power consumption can be changed to 4.1 W. In other words, if the upper part UCOL of the coil COL is far away from the core COE and adopts conductors of larger space distribution, then under the same stimulating strength, the total energy storage can be reduced to 79% and the power consumption can be reduced to 50%. This phenomenon is due to the fact that the AC resistance is mainly generated by the magnetic field, and if the same current is distributed in a larger space, then the power consumption can be further reduced (e.g., by adopting a wider conductor sheet or thicker conductors). Because the weight of the conductor having a larger cross-section area is greater, the design shown in FIG. 3F may be adopted by replacing the 8 conductor sheets of FIG. 3E with 24 conductor of FIG. 3F on the upper part UCOL of the coil COL, while the lower part LCOL of the coil COL remain at 8 conductors. That is, a conductor of the lower part LCOL of the coil COL extends out of the groove and is divided into 3 conductors and the 3 conductors are again incorporated into a conductor when it is winded back to the lower part LCOL of the coil COL. In FIG. 3F, the total width of the upper part UCOL of the coil COL in the space is 5.4 cm, the total height thereof is 1.2 cm and is still the same as that of FIG. 3E, and thus the total energy storage can be changed to 11 J, and the total power consumption is changed to 4.14 W. Moreover, another advantage of enlarging the equivalent cross-sectional area of the upper part UCOL of the coil COL is to reduce the leakage inductance, and to improve the coupling situation.

In another aspect, when the conductors of the lower part LCOL of the coil COL extend outward and are connected into a loop outside the core COE via the connecting part INT and the upper part UCOL of the coil COL, the number of the conductors of the lower part LCOL may be less than the number of the conductors of the upper part UCOL (e.g., be a half of the number of the conductors of the upper part UCOL). That is, a first number of conductors of the lower part LCOL of the coil COL is less than a second number of conductors of the upper part UCOL of the coil COL. Or, the multiple conductors in upper part UCOL can be substituted with a large cross-sectional area conductor instead. The present invention when under such configuration, not only being capable to reduce the DC resistance, but also reduce the proximity effect and the AC resistance further.

In other embodiments, a conductor spacing of the upper part UCOL of the coil COL is larger than a conductor spacing of the lower part LCOL so that a total conductor spacing of the upper part UCOL is larger than a total conductor spacing of the lower part LCOL of the coil COL to reduce the AC impedance.

In other embodiments, the upper part UCOL of the coil COL may be presented as distributed or branched, as shown in FIG. 4A to FIG. 4C. The distribution of the upper part UCOL of the coil COL may further reduce the summation of the magnetic field, thereby reducing the proximity effect and the AC impedance. The branched upper part UCOL of the coil COL may further reduce the total resistance. The thermal energy generated by the lower part LCOL of the coil COL can be directed to the upper part UCOL of the coil COL via conductors. The upper part UCOL of the coil COL has a very large contacting area with air, so it can effectively dissipate heat. It shall be appreciated that, FIG. 4A to FIG. 4C are only used to illustrate the structure of the coil COL, so the upper fastening component UFC and the lower fastening component LFC are not depicted therein.

Moreover, in other embodiments, the upper part UCOL of the coil COL may use conductors of a larger area, i.e., a conductor width of the upper part UCOL is larger than a conductor width of the lower part LCOL so that the cross-sectional area of the conductors of the upper part UCOL is larger than the cross-sectional area of the conductors of the lower part LCOL of the coil COL to reduce the AC impedance.

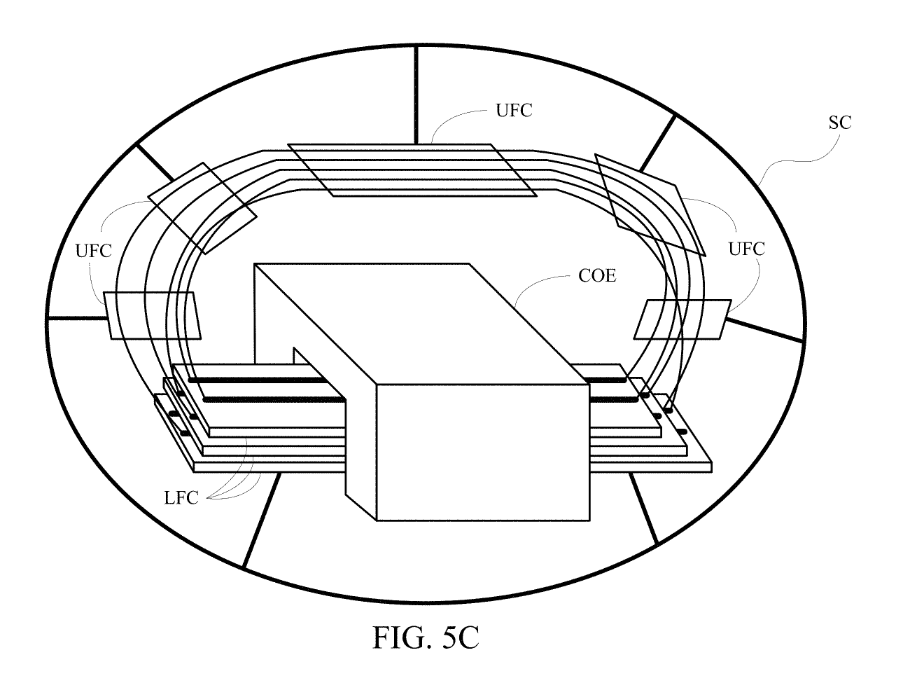

Referring to FIG. 5A to FIG. 5B, the upper fastening component UFC and the lower fastening component LFC are all insulators (e.g., plastic materials, ceramic materials) and are respectively used for fastening the upper part UCOL, the lower part LCOL and the connecting part INT of the coil COL, thereby preventing the conductors from contacting with each other due to intertwined coil. The number of conductors of the upper part UCOL of the coil COL may be greater than the number of conductors of the lower part LCOL, or sheet-shaped conductors or conductors of a larger diameter are adopted as the conductors, as a result, a larger area is required to serve as the upper fastening component UFC for maintaining the structure of the upper part UCOL of the coil COL, as shown in FIG. 5B.

In other embodiments, the inductor L further includes a supporting component SC, the supporting component SC is also an insulator and is winded around the inductor L, and the supporting component SC contacts with the upper fastening component UFC and the lower fastening component LFC to enhance the effect of maintaining the structure of the upper part UCOL and the lower part LCOL of the coil COL by the upper fastening component UFC and the lower fastening component LFC, as shown in FIG. 5C.

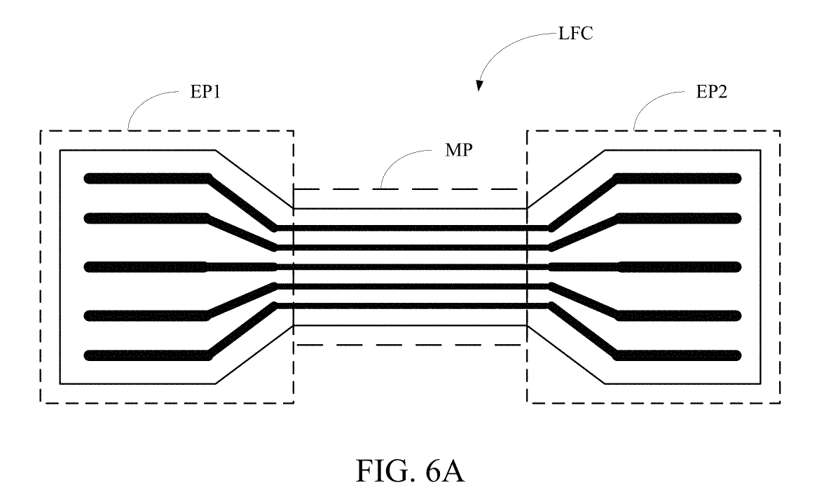

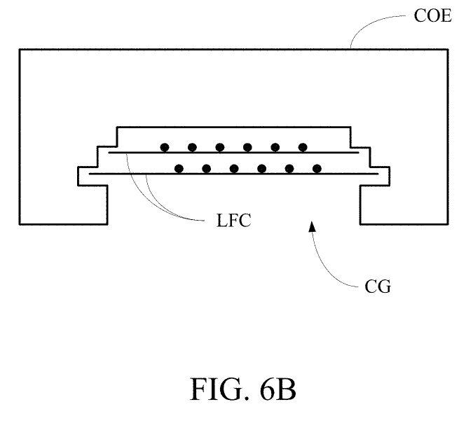

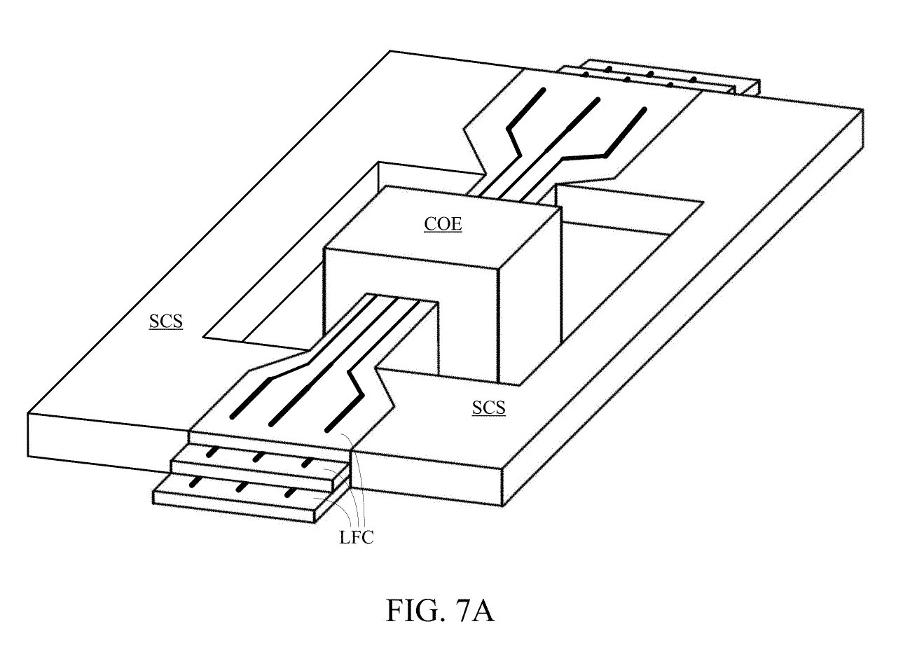

FIG. 6A depicts a schematic view of the lower fastening component LFC. The lower fastening component LFC includes a plurality of ceramic substrates, and each of the ceramic substrates has a middle part MP, a first end EP1 and a second end EP2. The first end EP1 and the second end EP2 are connected via the middle part MP. These ceramic substrates are stacked into a layered structure, and the connecting portion CP of the core COE shields the middle parts MP of the ceramic substrates. The lower fastening component LFC may be fastened within the groove of the core COE in a stepped or cogged manner, as shown in FIG. 6B.

Moreover, for any two adjacent ceramic substrates among the ceramic substrates CS, a size of an upper ceramic substrate is smaller than that of a lower ceramic substrate so that a ladder shape is formed respectively by the first ends EP1 and the second ends EP2 (but not limited thereto). For example, as shown in FIG. 1, in the lower fastening components LFC, the length of a second ceramic substrate is larger than the length of a first ceramic substrate, and the length of a third ceramic substrate is larger than the length of the second ceramic substrate. In this case, the length of the conductors on the first end EP1 and the second end of the upper ceramic substrate will be smaller than the length of the conductors on the first end EP1 and the second end of the lower ceramic substrate.

It shall be appreciated that, the shape of the lower fastening component LFC may vary according to requirements of circuit arrangement. For example, the lower fastening component LFC may be a ceramic substrate of a rectangular shape as shown in FIG. 1, or a dumbbell-shaped ceramic substrate as shown in FIG. 6A. Moreover, aluminium oxide, aluminium nitride, silicon nitride, silicon carbide and/or other thermal-conductive but not electrical-conductive ceramic materials may be used as the ingredients of the ceramic substrates. How to design the appearance and shape of the ceramic substrates and determine the material of the ceramic substrate according to different requirements of circuit arrangement shall be appreciated by those of ordinary skill in the art, and thus will not be further described herein.

The heat-dissipating speed of the inductor L can be improved by disposing the lower part LCOL of the coil COL on the ceramic substrate, so the inductor L will not be destroyed when the current on the coil COL is concentrated at a position in the space (e.g., the middle part MP of the ceramic substrate). A conductor width of the coil COL that is disposed at the first end EP1 and the second end EP2 may be larger than a conductor width of the coil COL that is disposed at the middle part MP. Moreover, the ceramic substrate and the lower part LCOL of the coil COL may be directly joined via a specific process (e.g., a thin film process, a thick film process, a direct bonding copper technology, a direct plating copper technology and an active metal hard welding technology or the like).

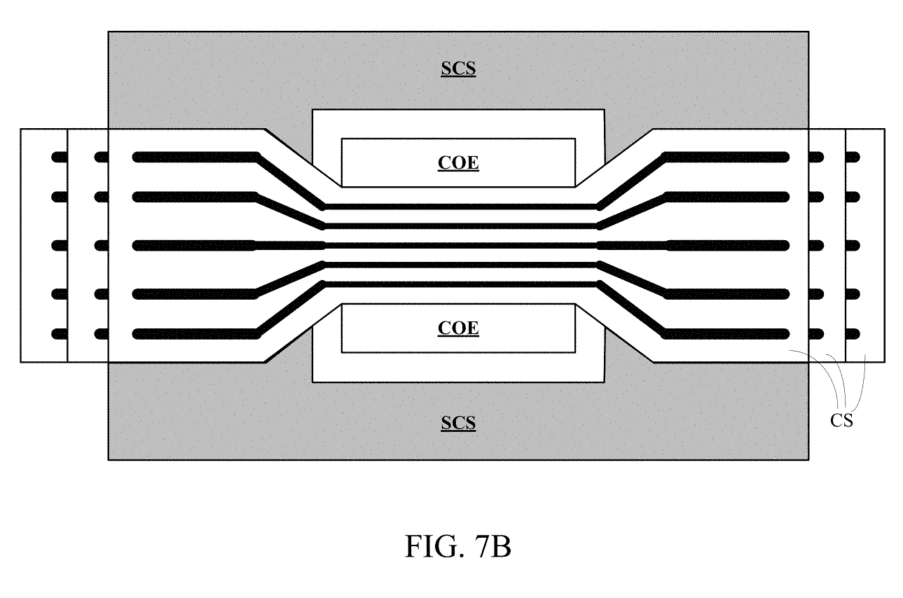

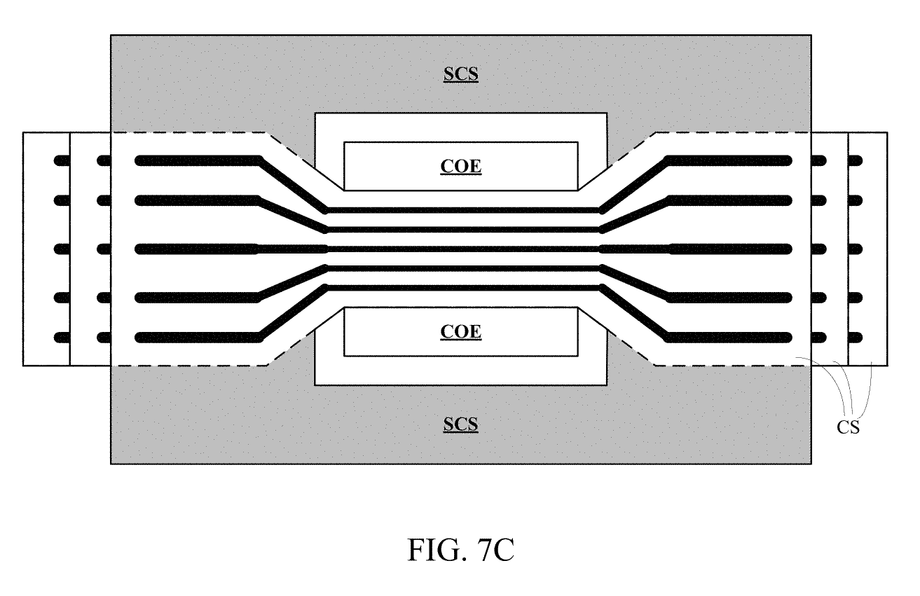

In other embodiments, the inductor L may further include a plurality of extended ceramic substrates SCS which, as shown in FIG. 7A, may be manufactured to have the same height as these lower fastening components LFC after being stacked and contact with the lower fastening components LFC. The extended ceramic substrate SCS may also be manufactured to contact with the lower fastening components LFC respectively along the two side portions SP of the core COE, as shown in FIG. 7B. Moreover, when the lower fastening components LFC are ceramic substrates, these extended ceramic substrates SCS may be further designed to be formed integrally with the ceramic substrates, as shown in FIG. 7C. Moreover, in another embodiment, the extended ceramic substrates SCS may be manufactured to have the same thickness with the lower fastening components LFC or become a part of the lower fastening components LFC (i.e., formed integrally with the lower fastening components LFC), and stacked sequentially along with the lower fastening components LFC.

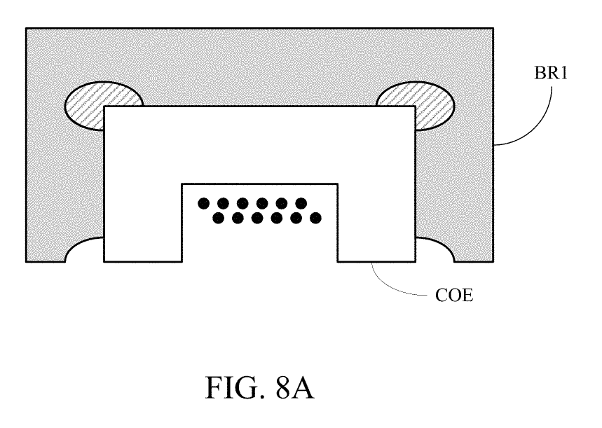

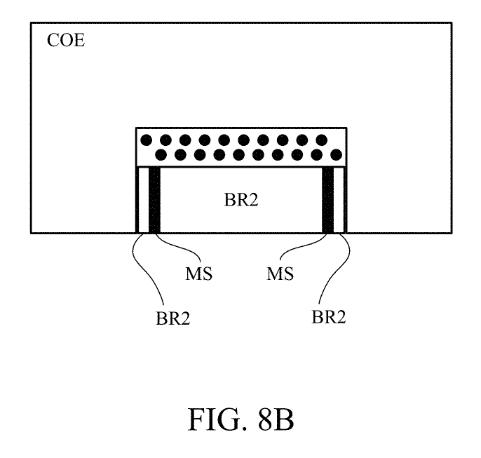

In other embodiments, the inductor L further includes an upper blocking component BR1, and the core COE of the inductor L is at least partly covered by the upper blocking component BR1, as shown by the grey part of FIG. 8A. The upper blocking component BR1 may reduce the noise generated by the inductor L during the use thereof. The upper blocking component BR1 is made of a material of high hardness, e.g., ceramic, silicon carbide, aluminum oxide or the like. The material of high hardness is a substance of which the young's modulus is at least greater than 50 GPa (gigapascal). Moreover, the upper blocking component BR1 further comprises part cavities (i.e., the part indicated by slash lines in FIG. 8A), and the cavities may prevent the breakage of the upper blocking component BR1 due to the vibration of the inductor L generated during the operation thereof.

In other embodiments, the inductor L further includes a lower blocking component BR2 and a magnetostrictive material MS, and the lower blocking component BR2 and the magnetostrictive material MS are disposed at the inner sides of two side portions of the core COE, as shown in FIG. 8B. The magnetostrictive material is a substance with a magnetostriction ratio greater than 100 ppm. Specifically, deformations would appear during the operation of the rTMS apparatus 1 due to the Lorent force, the reluctance force and the magnetostriction, and they would cause the core COE and the coil COL to vibrate and generate noise. The coil COL is mainly influenced by the Lorentz force, while the core COE is mainly influenced by deformations caused by the reluctance force and magnetostriction.

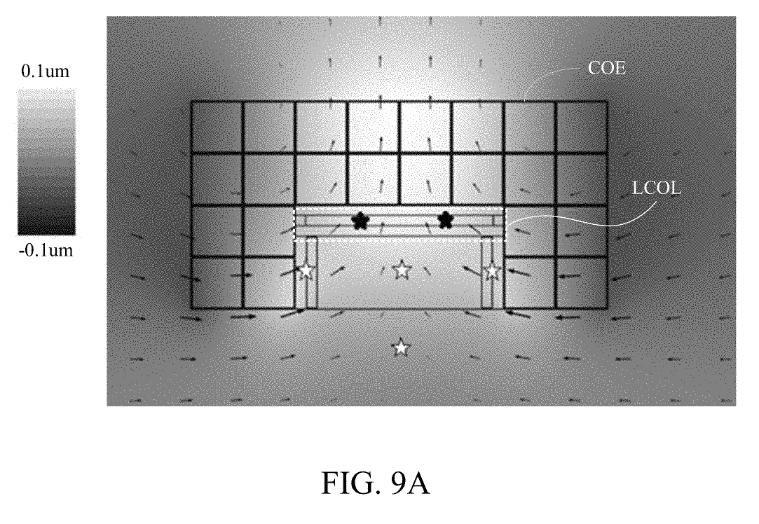

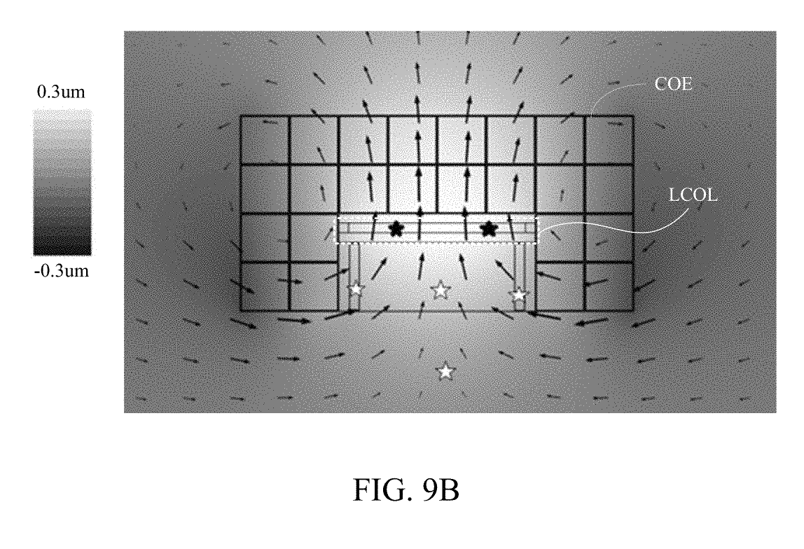

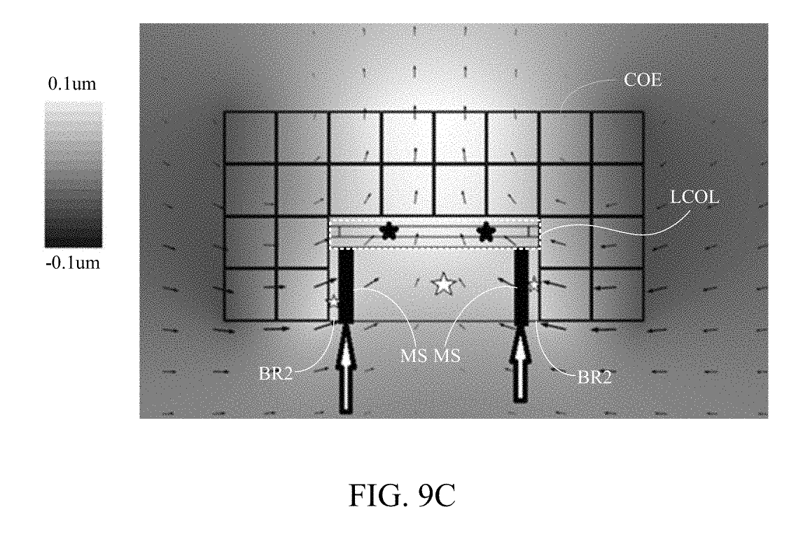

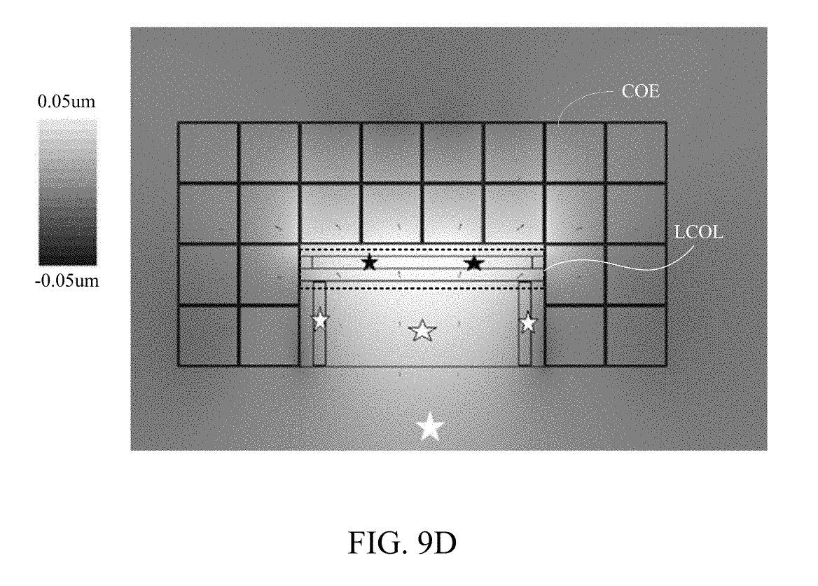

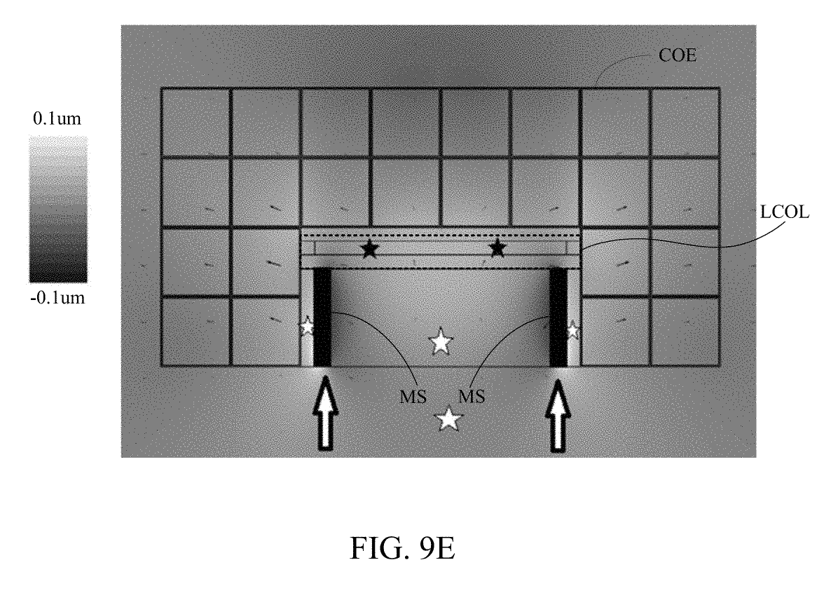

For the magnetic field intensity having a value of 0.2 T at the depth of 2 cm, when only the reluctance force to the core COE and the Lorentz force to the coil COL are taken into consideration and the magnetostriction is not taken into consideration, the directions of the deformations are as shown by the directions of the arrows in FIG. 9A. Taking the case where the young's modulus of the core COE is equal to 200 Gpa and the ambient air is 2 GPa for calculation, the upward deformation from the central portion (i.e., the connecting part CP) of the core COE is 0.14 um, and the inward deformations from the two side portions SP are 0.2 um. It shall be appreciated that, the young's modulus of the ambient air is set to 1% of the young's modulus of the core COE for preventing the calculation result being influenced; hence, it is not the real hardness.

In the case where the reluctance force of the core COE, the Lorentz force of the coil COL and the magnetostriction are taken into consideration at the same time, the directions of the deformations are as shown by the directions of the arrows in FIG. 9B. If the lambda of the core COE is set to be 3e-5, the saturation magnetization is set to be 2e6, then it can be observed that the upward deformation from the central portion of the core COE is 0.22 um, the inward deformations from the two side portions SP are still 0.2 um, and the direction of the deformation is different from that of FIG. 9A. In other words, the deformation caused by part reluctance force and the deformation caused by the force of magnetostriction may be added or canceled with each other because the directions thereof may not be the same, thereby causing different deformations in shape.

In other embodiments, the inductor L further includes a lower blocking component BR2, and inner sides of the two side portions SP of the core COE are connected with the lower blocking component BR2, as shown in FIG. 9C, and the lower blocking component BR2 can reduce the noise generated by the inductor L during the operation. The lower blocking component BR2 is made of a material of high hardness, e.g., ceramic, silicon carbide, aluminum oxide or the like. The lower blocking component BR2 may include a magnetostrictive material MS other than the core COE. As shown in FIG. 9C, the magnetostrictive material MS is added at the inner sides of the two side portions SP of the core COE, the lambda thereof is 2e-3, the saturation magnetization is 1e6, the young's modulus is 30 Gpa, the upward deformation from the central portion of the core COE can be further set to be 0.02 um, and the inward deformations of the two side portions SP are 0.07 um.

In other embodiments, the strength of the surrounding structure may be enhanced to reduce the vibration. For example, if the young's modulus of the upper blocking component BR1 and the lower blocking component BR2 is changed into 200 Gpa, then the upward deformation from the central portion of the core COE is 0.006 um, and the inward deformations of the two side portions SP are 0.013 um, as shown in FIG. 9D. Moreover, for the case where the magnetostrictive material MS is added, if the young's modulus of the upper blocking component BR1 and the lower blocking component BR2 is changed into 200 Gpa, then the upward deformation from the central portion is 0.018 um, and the inward deformations of the two side portions SP are 0.03 um, as shown in FIG. 9E. The thickness of the upper blocking component BR1 should range from 2 mm to 20 mm to match the thickness of the core.

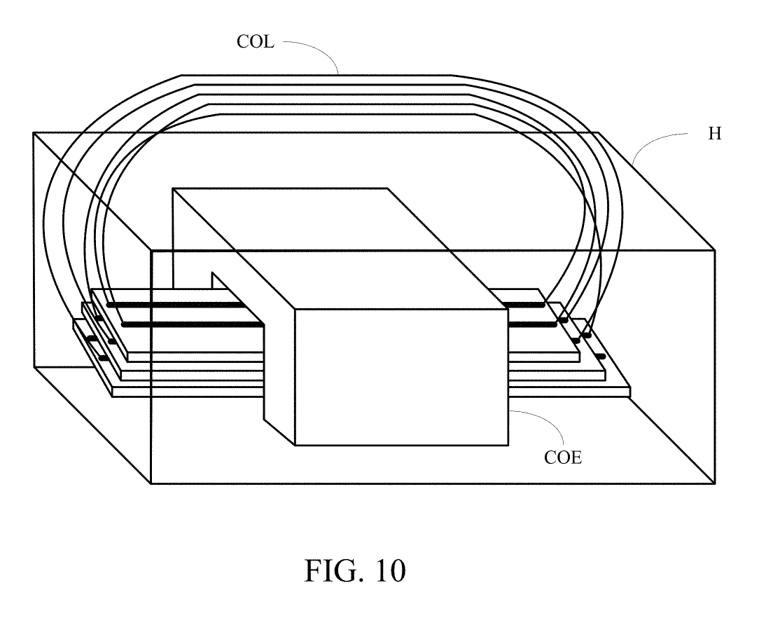

In other embodiments, the rTMS apparatus 1 further comprises a housing H, and the inductor L is disposed within the housing H, as shown in FIG. 10. The material of the housing H has high acoustic impedance, and the housing H at least covers main vibration sources such as the core COE and the lower part LCOL of the coil COL in the space, thereby reducing the transmission of noise. At least one layer of material having low acoustic impedance (e.g., air, porous sound absorbing materials or the like) is used between the housing H and the inductor L to increase the reflection. The core COE, the lower part LCOL of the coil COL and a part of the connecting portion INT are located within the housing H. A spring with a specific damping and elastic coefficient used between the inductor L and the housing H may make the self-vibration frequency of the inductor L which is generated from the current, and the vibration frequency of combined inductor L and the housing H, being separated. Therefore, the sound wave generated by the housing H is reduced. Moreover, the housing H and the inductor L are only connected via the specific spring and damp without other contact. Therefore, when a part of the upper part UCOL of the coil COL is disposed outside the housing H, the connection of the connecting portion INT of the coil COL and the housing H should be taken into consideration when considering elastic coefficient and damping. Moreover, if a porous sound absorbing material is filled between the housing H and the core COE, then the porous sound absorbing material also needs to be taken into consideration.

Moreover, the sound wave generated by the core COE and the conductor is reflected back into the cavity (i.e., the space between the inductor L and the housing H) when it encounters high acoustic impedance, and is oscillated in the cavity and finally absorbed. The spacing between the inductor L and the housing H may be designed according to the one-quarter-wavelength principle. For example, the wavelength corresponding to the frequency of 10 kHz is about 3.4 cm, so adopting a cavity having a one-quarter wavelength of 0.85 cm and filling the porous sound absorbing material or other sound absorbing materials can help to transform the sound wave oscillated in the cavity into thermal energy and make it finally being absorbed. Additionally, in other embodiments, the upper part UCOL of the coil COL may also be completely disposed within the housing H. The housing H and the core COE are separated by at least a layer of substance having low acoustic impedance therebetween. The housing H and the core COE are joined by a spring. An elastic coefficient of the spring ranges from 0.001 mm/N to 0.1 mm/N. A thickness of the housing H ranges from 0.5 mm to 1 cm. A thickness of the substance having low acoustic impedance ranges from 0.4 cm to 8 cm. The substance having low acoustic impedance is defined as a substance below 0.01 MPa*s/m³, and a substance having high acoustic impedance is defined as a substance ranging from 1 to 100 MPa*s/m³. The implementation of this aspect shall be appreciated by those of ordinary skill in the art, and thus will not be further described herein.

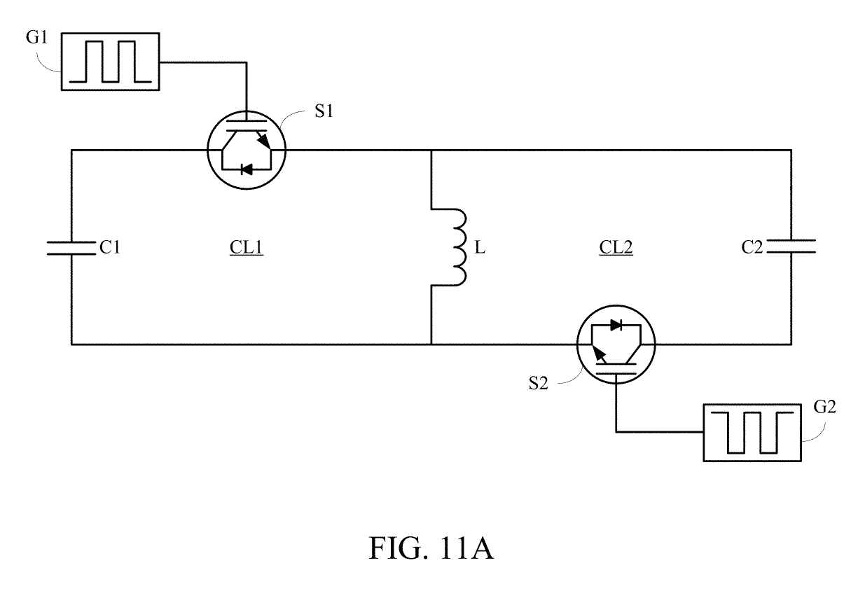

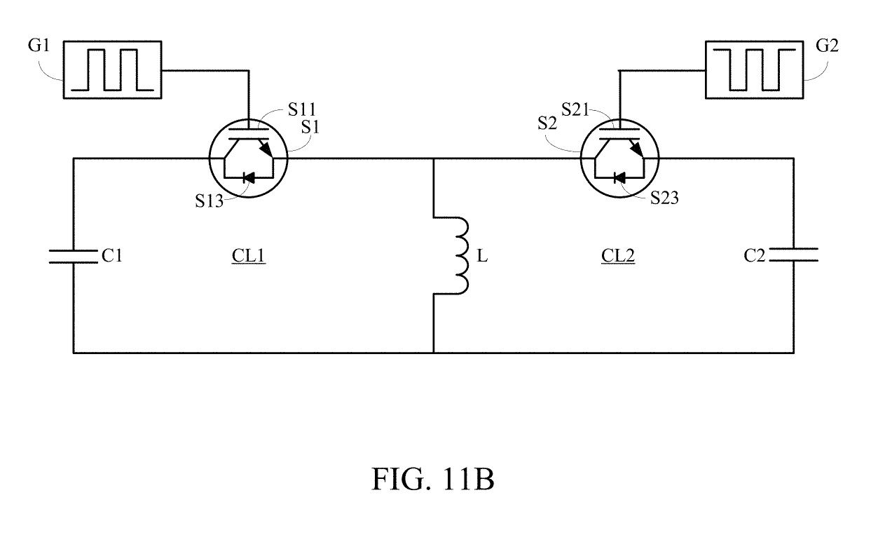

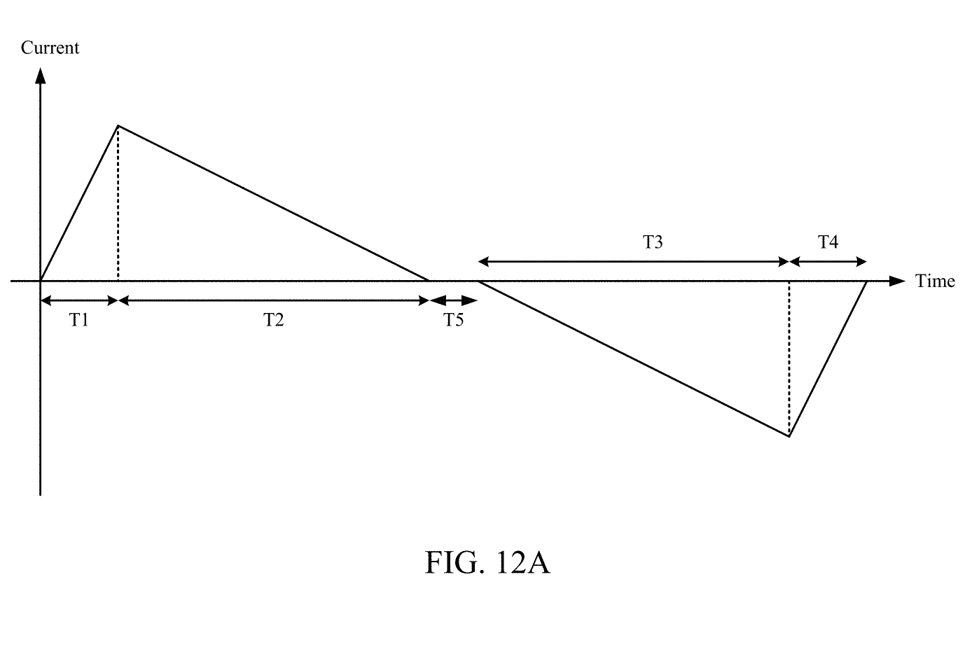

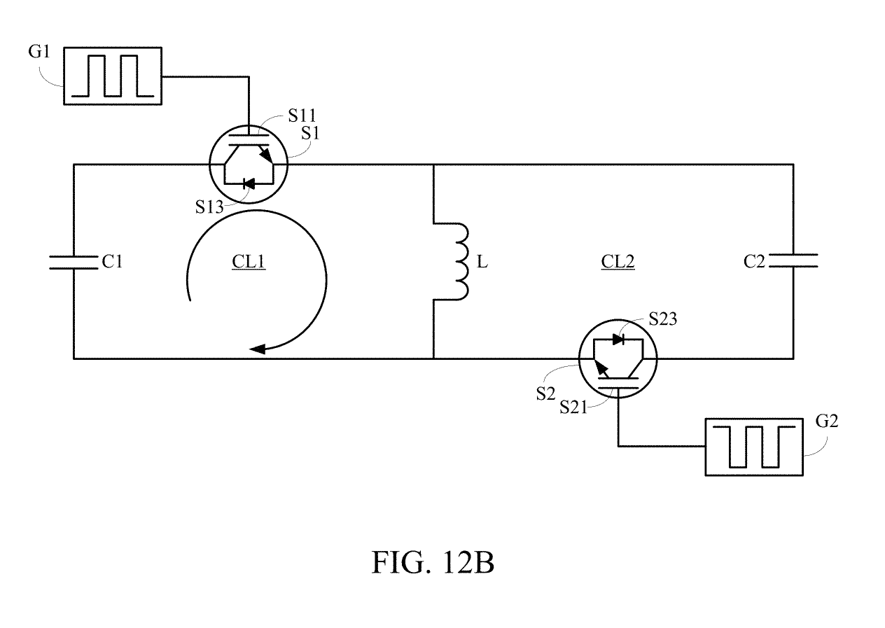

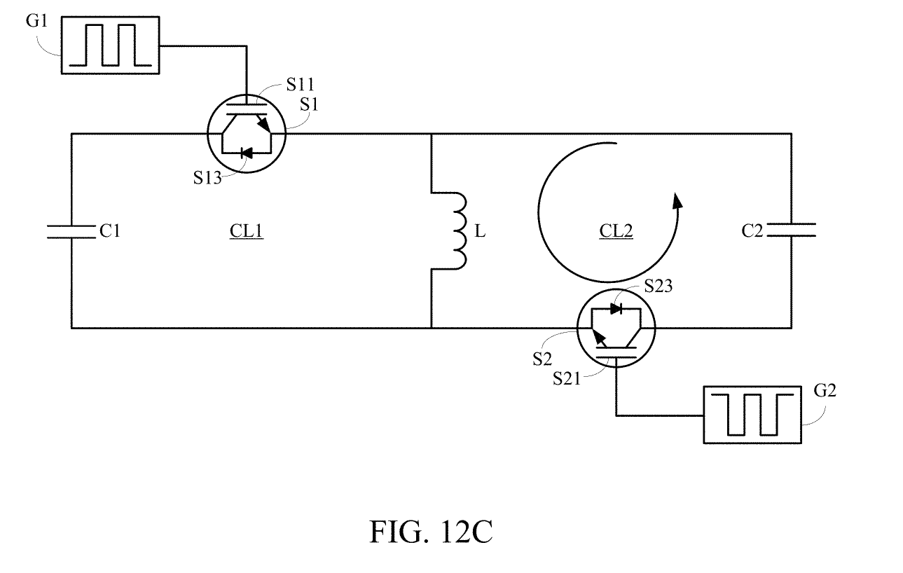

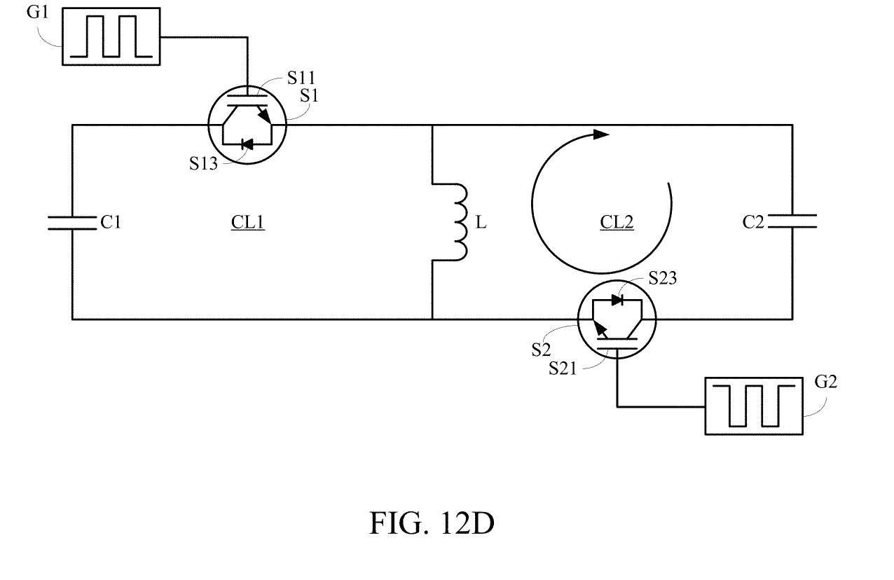

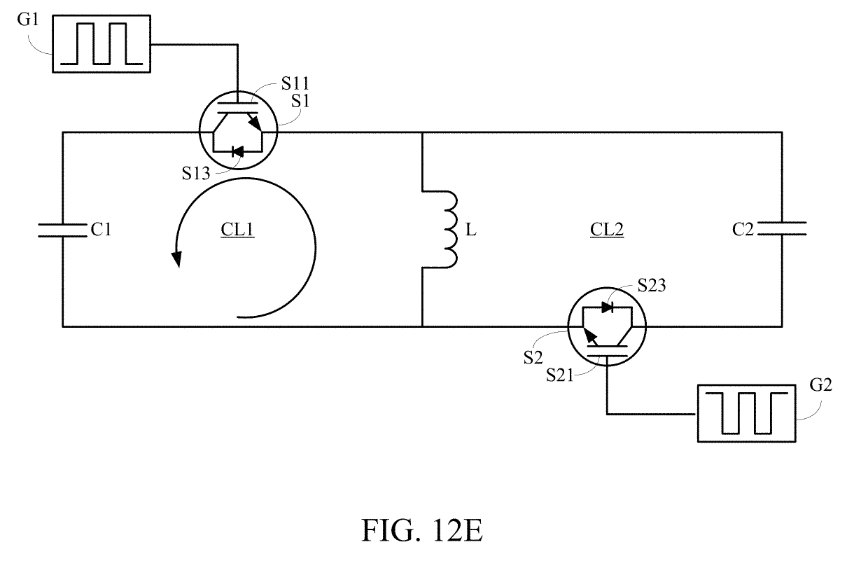

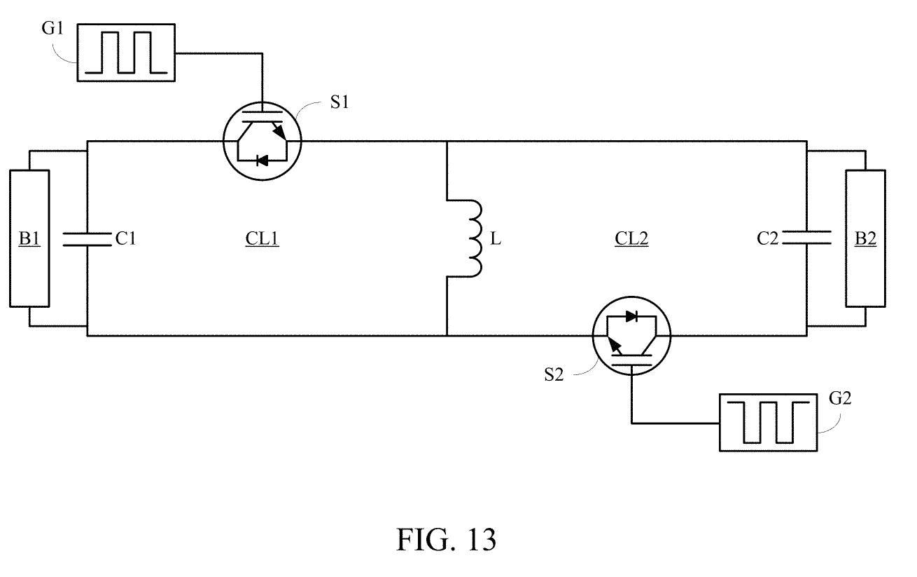

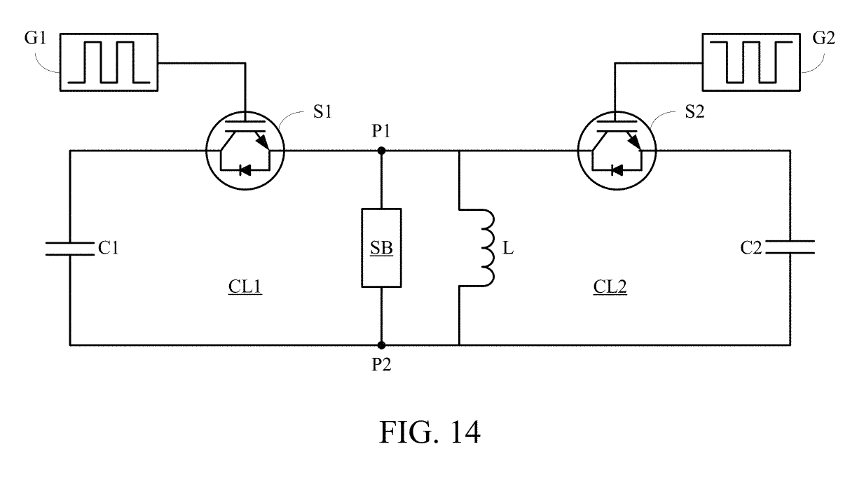

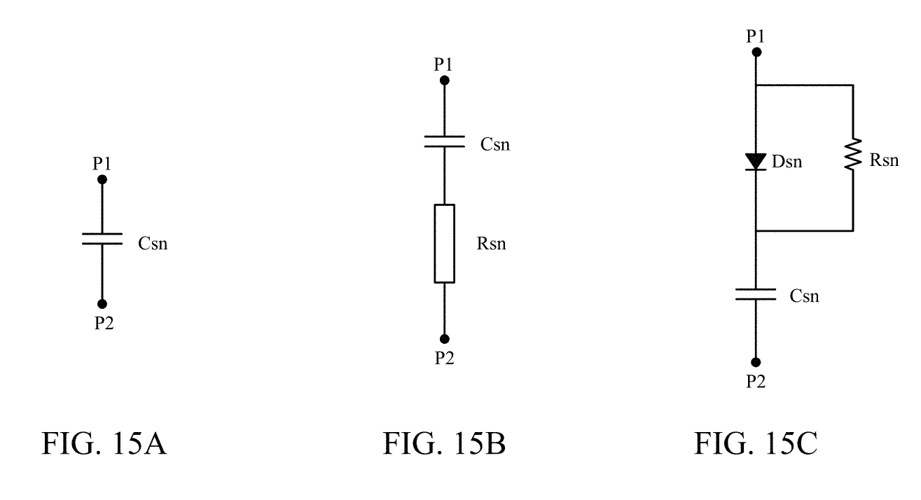

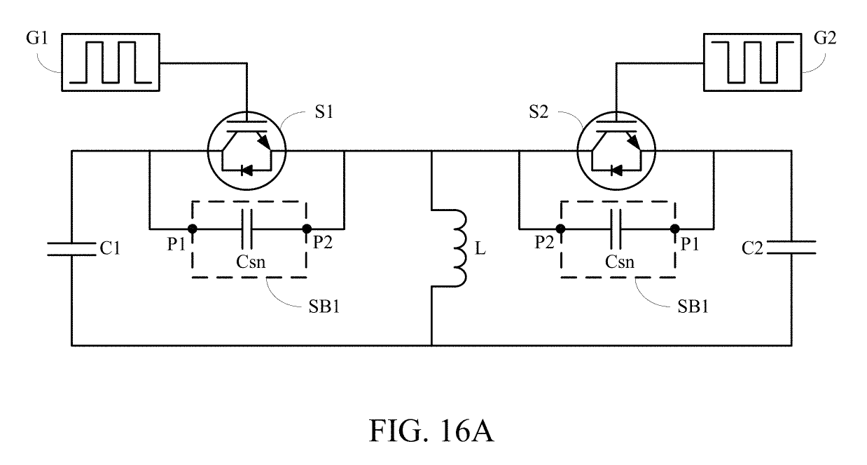

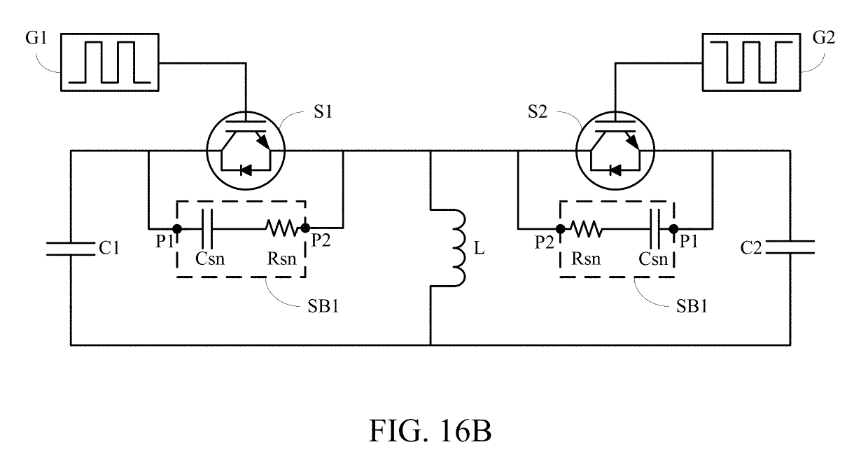

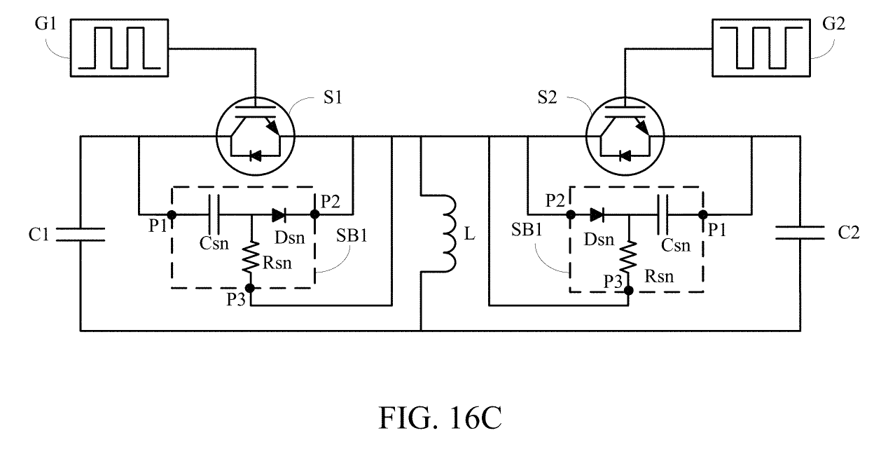

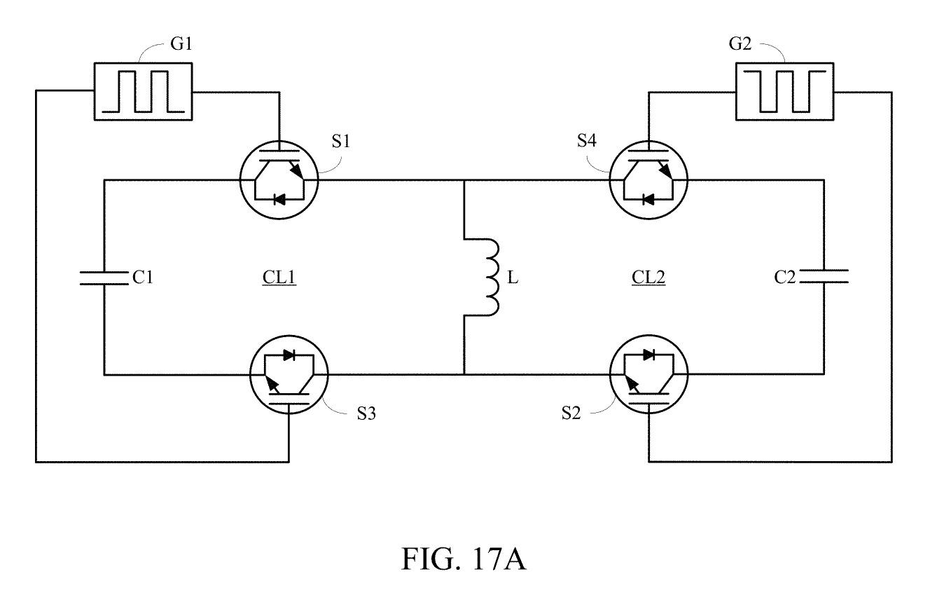

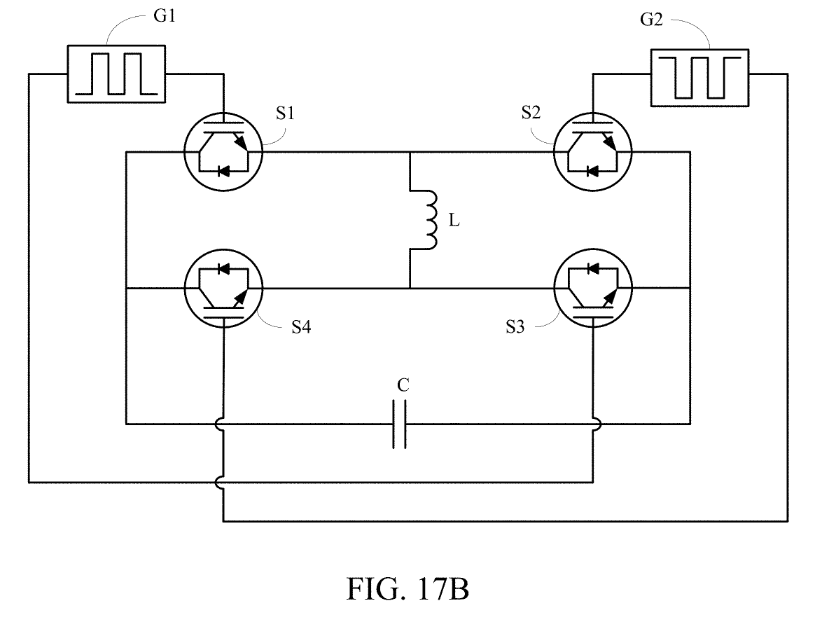

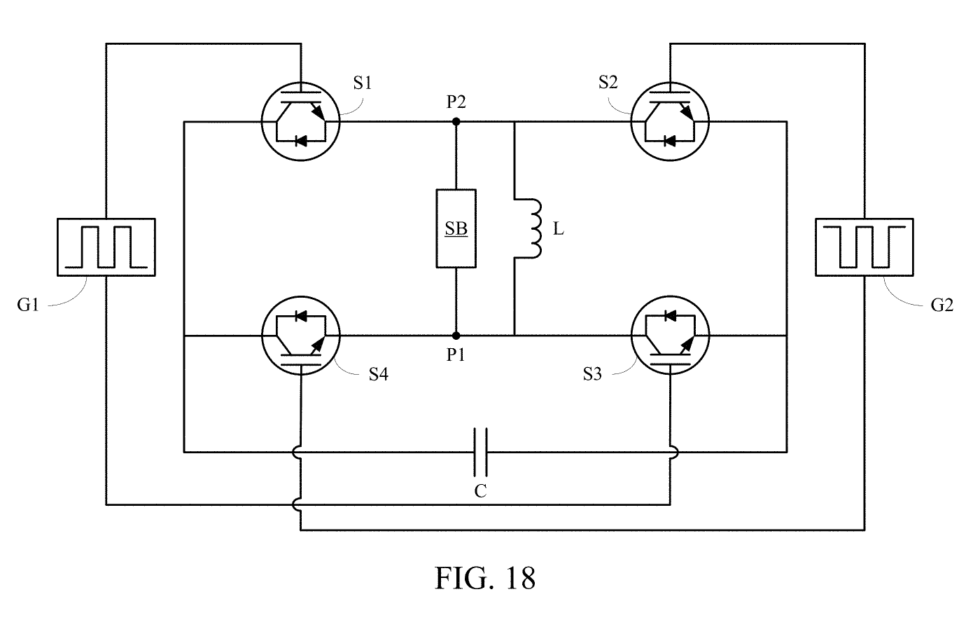

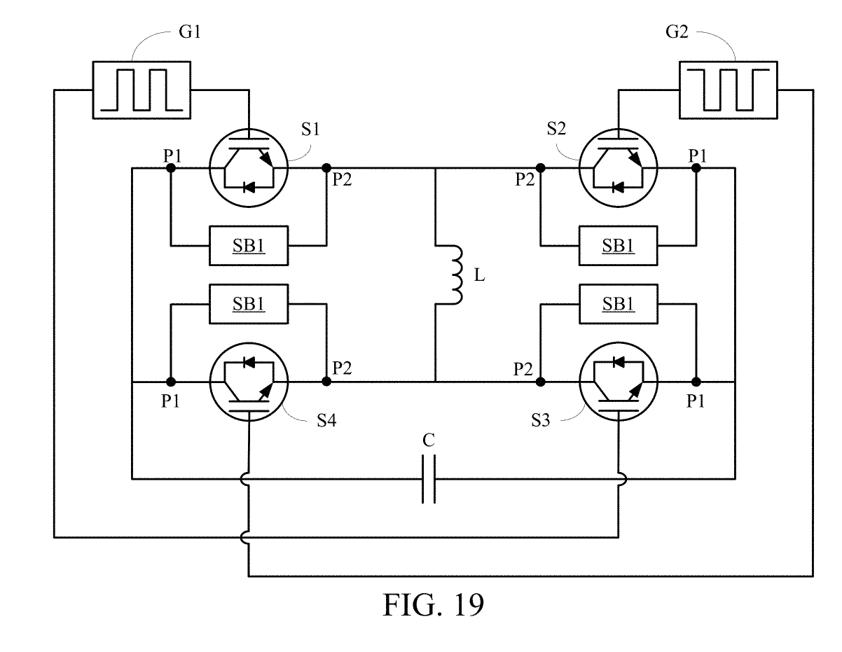

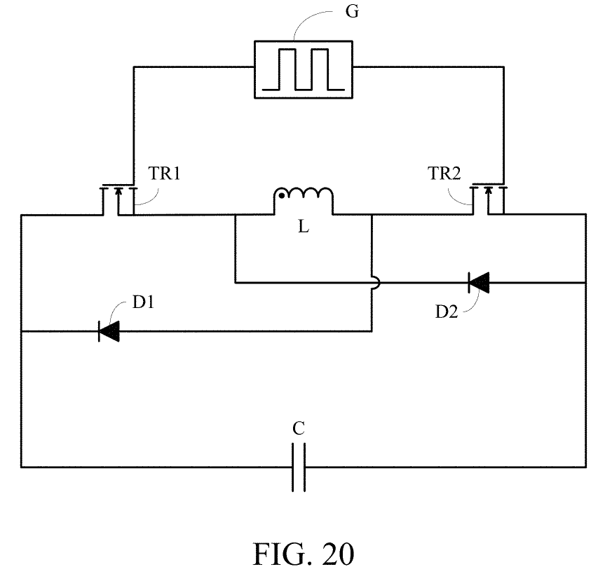

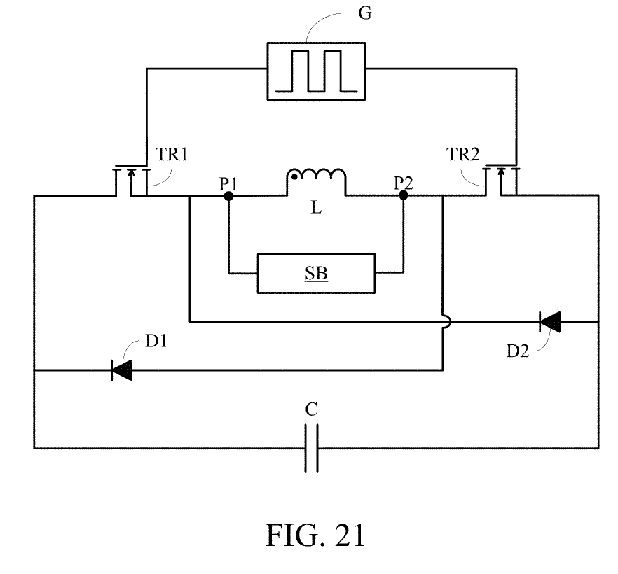

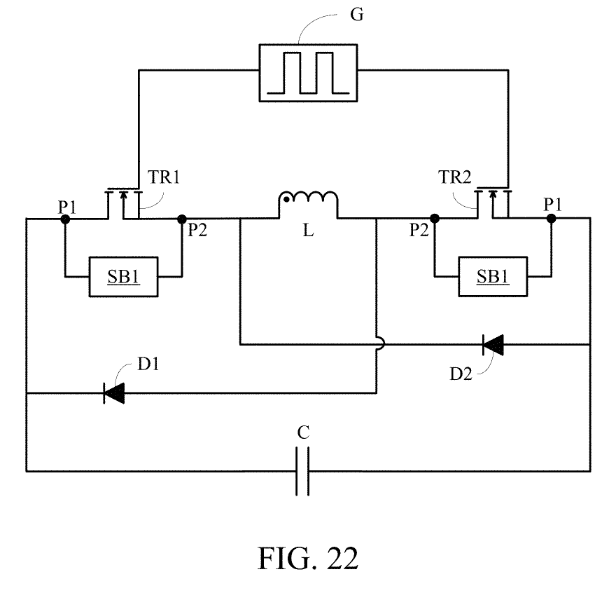

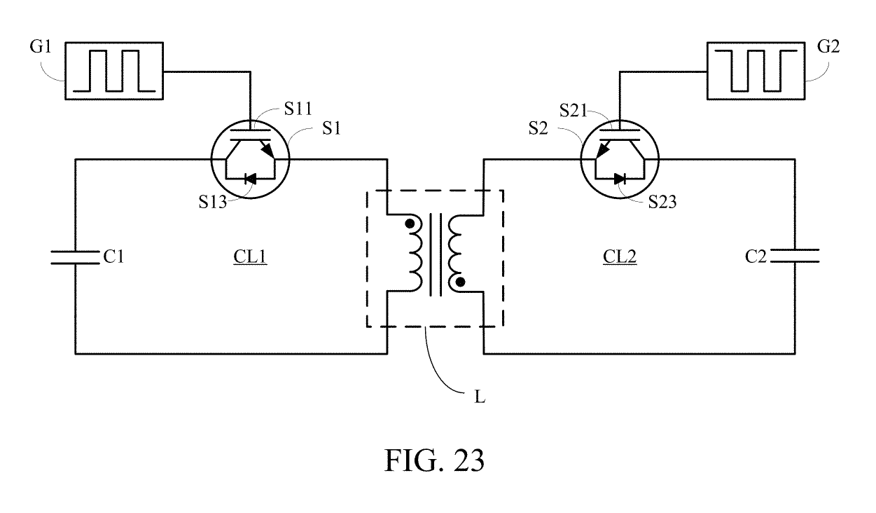

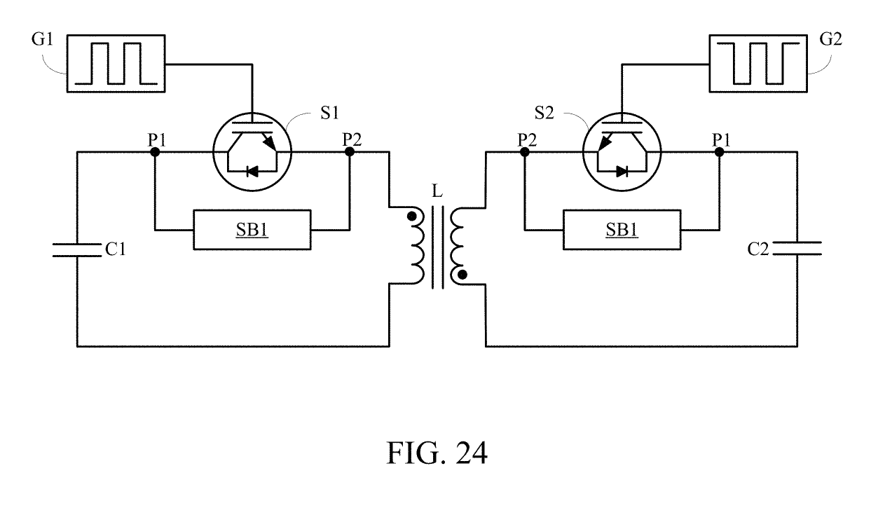

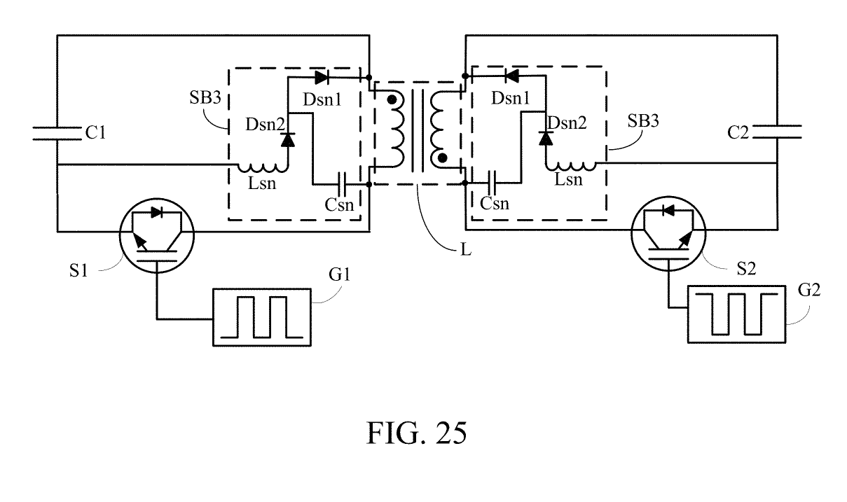

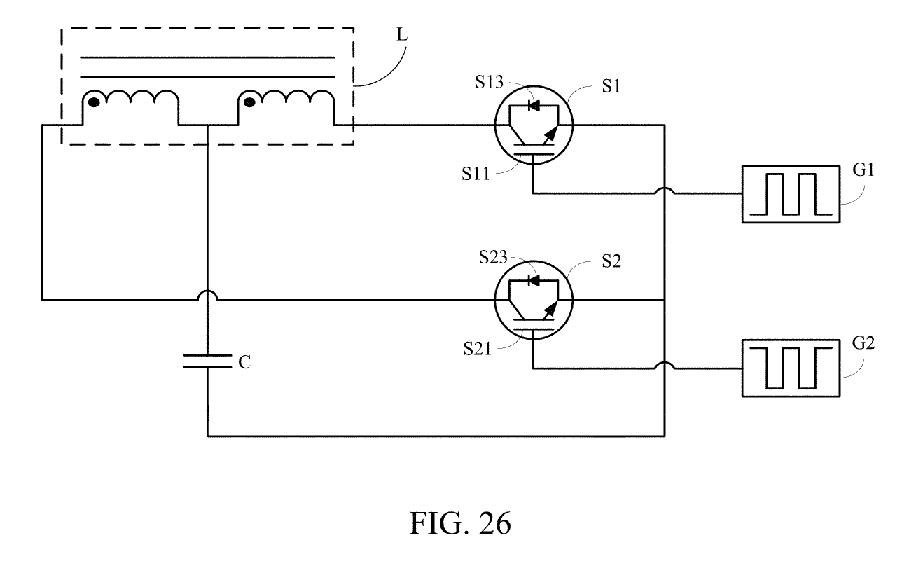

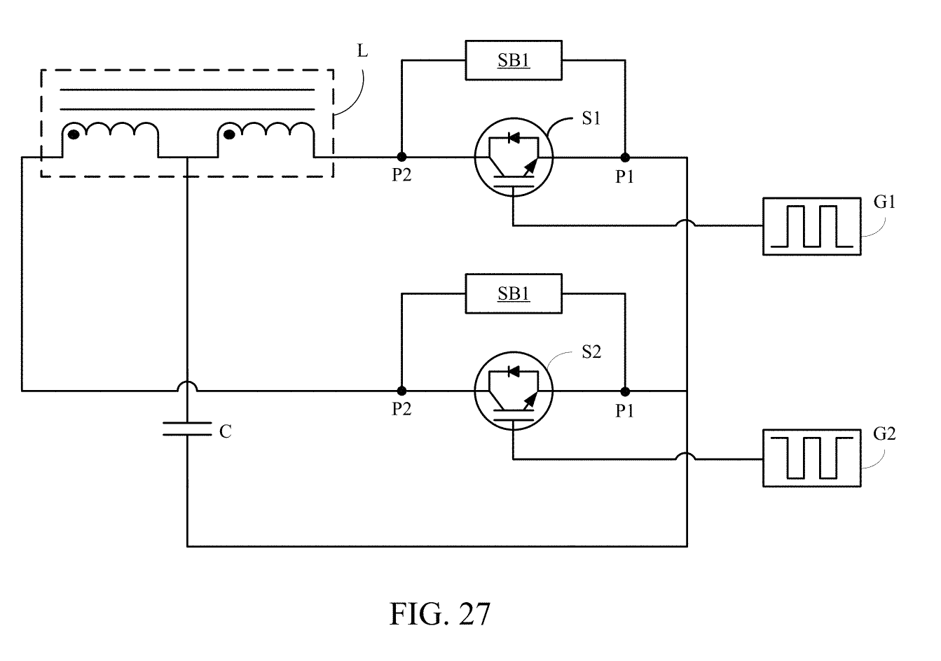

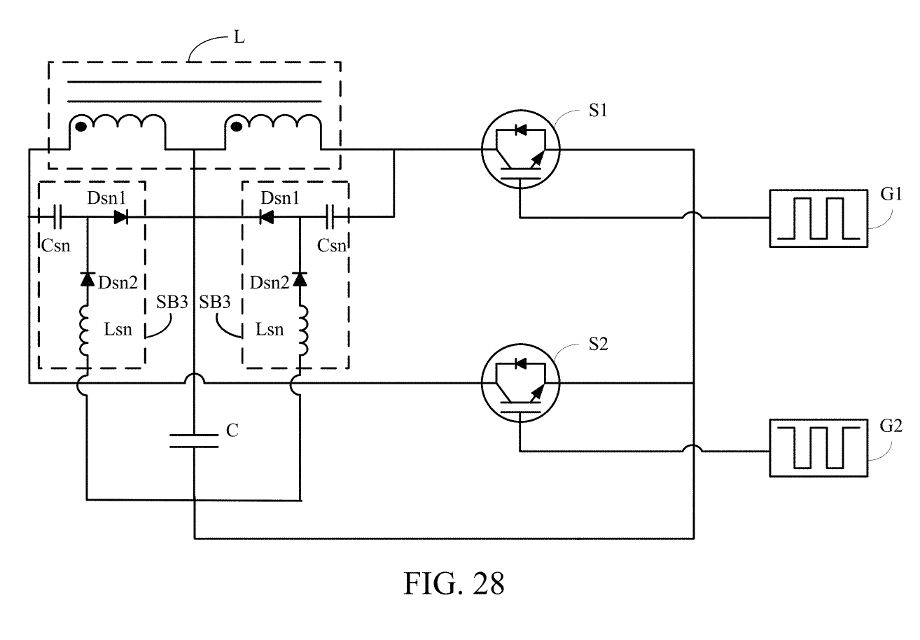

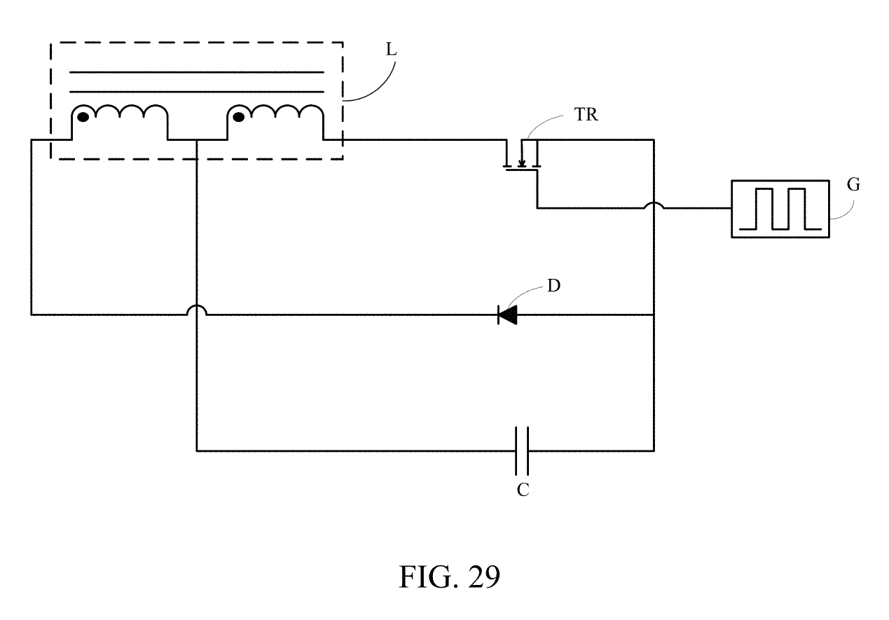

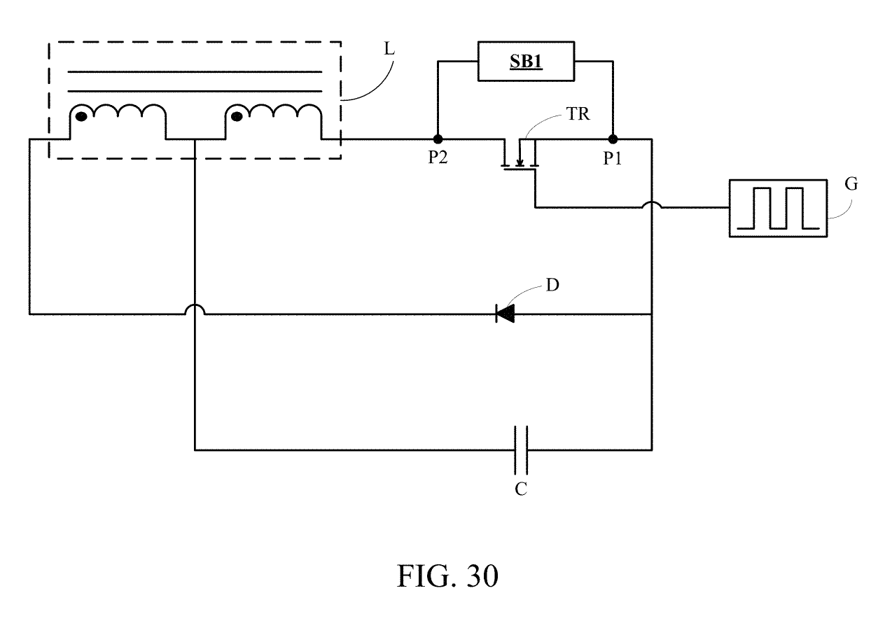

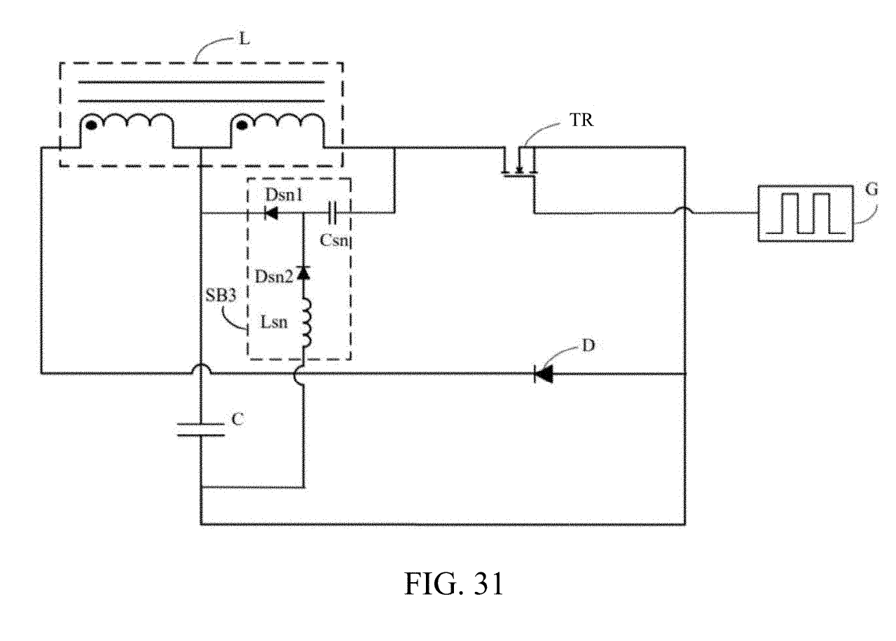

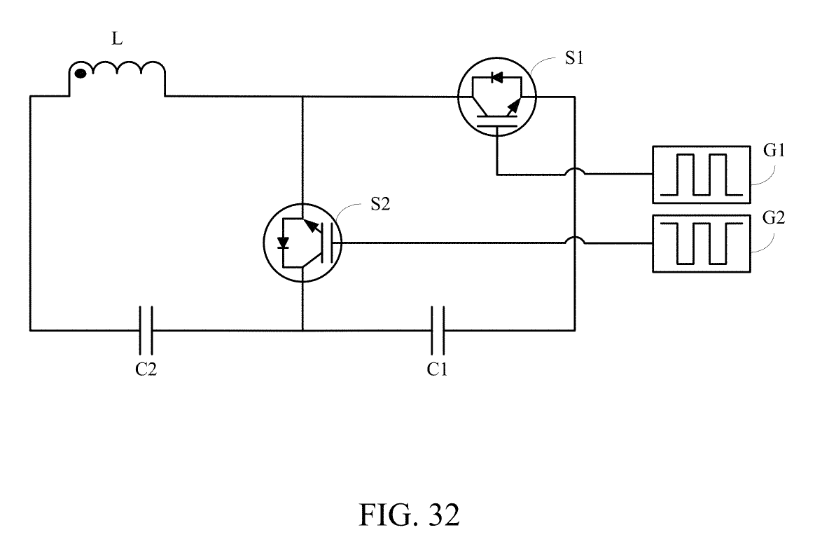

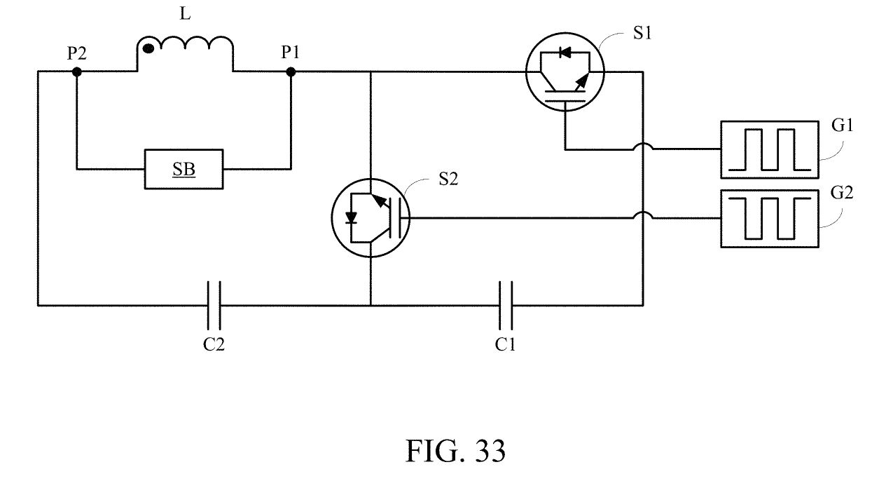

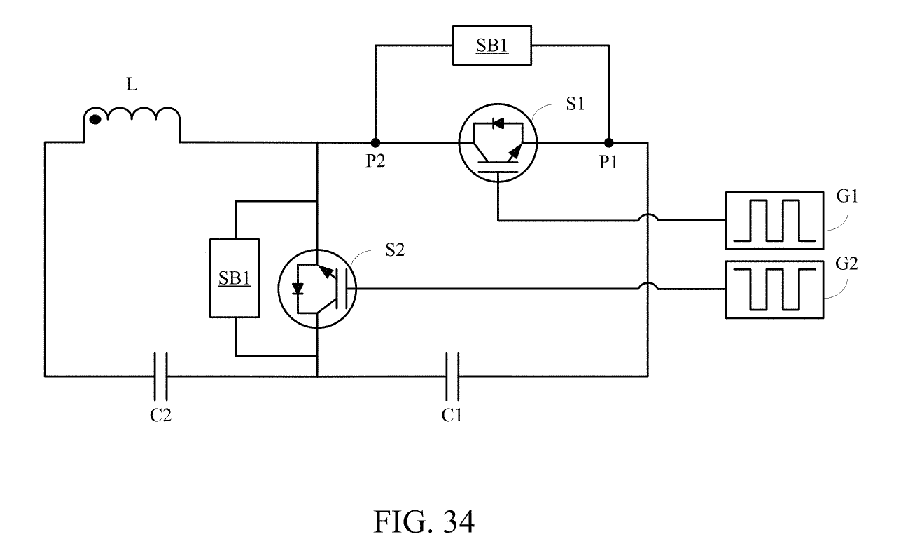

A second embodiment of the present invention is as shown in FIG. 11A to FIG. 11B, FIG. 12A to FIG. 12E, FIG. 13 to FIG. 14, FIG. 15A to FIG. 15C, FIG. 16 and FIG. 17A, and it is an extension of the first embodiment. FIG. 11A and FIG. 11B are schematic views depicting the circuits of the portable rTMS apparatus 1 of the present invention. The driver circuit DRC comprises a first capacitor C1, a second capacitor C2, a first switch with freewheeling diode S1, a second switch with freewheeling diode S2, a first pulse generator G1 and a second pulse generator G2.

The first energy storage capacitor C1, the first switch with freewheeling diode S1 and the inductor L are connected in series to form a first driver circuit loop CL1. The second energy storage capacitor C2, the second switch with freewheeling diode S2 and the inductor L are connected in series to form a second driver circuit loop CL2. The aforesaid circuit is connected via a half-bridge primary side. It shall be appreciated that, the schematic views of the circuits in FIG. 11A to FIG. 11B are not intended to limit the sequence of the first energy storage capacitor C1 and the first switch with freewheeling diode S1 in the first driver circuit loop CL1 and the sequence of the second energy storage capacitor C2 and the second switch with freewheeling diode S2 in the second driver circuit loop CL2. In practical application, the provider or the manufacturer of the portable rTMS apparatus 1 may decide the order in which the capacitors and the switches are connected in series according to different requirements.

The first driver circuit loop CL1 comprises the first pulse generator G1 that is coupled to the first switch with freewheeling diode S1 and generating a first pulse signal to control the first switch with freewheeling diode S1. The second pulse generator G2 is coupled to the second switch with freewheeling diode S2 and generating a second pulse signal to control the second switch with freewheeling diode S2. The first switch with freewheeling diode S1 has a controllable switch S11 and a diode S13 and the second switch with freewheeling diode S2 has a controllable switch S21 and a diode S23.

The switch with freewheeling diode is defined as an electrical network that is equivalent to a controllable switch connected in parallel with a reverse diode on the scale of circuit in household power suppliers. The switch with freewheeling diode can turn on bidirectional current when a control signal is activated, and it can turn on the reverse current or block the forward voltage when the control signal is deactivated. Because embodiments using the switch with freewheeling diode all have two loops causing different magnetic flux directions, the diode of the second driver circuit loop CL2 is immediately turned on when the first driver circuit loop CL1 is turned off, and thus it is called a freewheeling diode. The switches with freewheeling diode mentioned in the subsequent embodiments are all defined in the same way.

For example, the switch with freewheeling diode may be constituted in the following way: (1) an IGBT connected in parallel with a reverse diode; (2) a single metal oxide semiconductor field effect transistor; and (3) a metal oxide semiconductor field effect transistor first connected in series with a forward diode and then connected in parallel with a reverse diode, but not limited thereto. Practically, the switches may include various components so that the switches are equivalent to an electrical network formed by a controllable switch connected in series with a reverse diode when they are performing the operations of FIG. 11A to FIG. 11B and FIG. 12B to FIG. 12E.

The controllable switches S11 and S21 may be insulated gate bipolar transistors (IGBTs), metal oxide semiconductor field effect transistors (MOSFETs), gate turn-off thyristors (GTOs), bipolar junction transistors (BJTs), power MOSFETs, MOS-controlled thyristors (MCTs), integrated gate-commutated thyristors (IGCTs), injection enhanced gate transistors (IEGTs) or the like, but not limited thereto.

In other embodiments, the portable rTMS apparatus 1 of the present invention may further comprise a controller to control the first pulse generator G1 and the second pulse generator G2. The inductor L is used as a stimulator. The first pulse signal and the second pulse signal enable the first driver circuit loop CL1 and the second driver circuit loop CL2 to conduct alternately. The first driver circuit loop CL1 and the second driver circuit loop CL2 have magnetic flux driving directions opposite to each other in respect to the inductor L. The portable rTMS apparatus 1 of the present invention can generate asymmetrical waveforms by configuration of two energy storage capacitors (i.e., the first energy storage capacitor C1 and the second energy storage capacitor C2) and two freewheeling diode switches (i.e., the first switch with freewheeling diode S1 and the second switch with freewheeling diode S2), thereby achieving a better stimulating effect.

As described previously, the core COE of the inductor L of the present invention may be a high saturation flux density material to reduce the magnetic resistance (by about 75%) and reduce the current amount (by about 75%) required for generating the magnetic field of the same magnitude. In this case, because the reduction of the current amount may lead to the reduction of the energy stored in the inductor L, the present invention may use capacitors having smaller capacitance as the first energy storage capacitor C1 and the second energy storage capacitor C2. The energy storage capacitors are capacitors having an energy storage value greater than 2.5 J, and they are often called bulk capacitors on the scale of circuit in household power suppliers. Practically, the energy required by the inductor is about 25 J. The bulk capacitor is responsible for providing most of the energy supply during the operations as shown in FIG. 11 to FIG. 12, and the capacitance thereof is at least ten times greater than other non-energy storage capacitors on the circuit, e.g., the capacitor of a snubber. The capacitor may be formed by more than one physical capacitor connected in parallel or in series so that the capacitor is still equivalent to a bulk capacitor during the operation of the circuit. For example, a power supplier often uses many small capacitors connected in parallel in order to reduce the impedance of the conductor. The energy storage capacitors mentioned in the subsequent embodiments are all defined in the same way. In practical circuit design, a ratio of a first initial voltage of the first energy storage capacitor C1 to a second initial voltage of the second energy storage capacitor C2 may be substantially set to be about 4, and a ratio of a first working period of the first pulse signal to a second working period of the second pulse signal may also be substantially set to be about 4. However, as shall be appreciated by those of ordinary skill in the art, the aforesaid ratios may also be set to range from 1 to 10.

For example, referring to FIG. 12A to FIG. 12E, it is assumed that the inductance of the inductor L is 200 pH, the capacitance of the first energy storage capacitor C1 is 2.5 μF, the first initial voltage is 3600 V, the turn-on time of the first pulse generator G1 is 50 μs, and the capacitance of the second energy storage capacitor C2 is 40 pF, the second initial voltage is 700 V, the turn-on time of the second pulse generator G2 is 200 μs, and the delay time is 0.001 s. The first initial voltage represents that the first energy storage capacitor C1 is at the saturated status, and the second energy storage capacitor C2 is at the half-saturated status. When the first pulse signal generated by the first pulse generator G1 controls the controllable switch S11 within the first switch with freewheeling diode S1 to be turned on within a time interval T1, the first energy storage capacitor C1 is equivalent to a voltage source and starts to discharge so that the first driver circuit loop CL1 is turned on, and the direction of the current loop after turned on is clockwise, and the current value of the inductor L increases gradually within the time interval T1, as shown in FIG. 12A and FIG. 12B.

When the time interval T1 ends, the first pulse signal generated by the first pulse generator G1 controls the controllable switch S11 within the first switch with freewheeling diode S1 to be turned off, and the first driver circuit loop CL1 is turned off instantly. In response to this, the inductor L serves as a current source in the circuit within a time interval T2 and starts to discharge and the current value thereof decreases gradually within the time interval T2. Within the time interval T2, the discharge voltage of the inductor L enables a diode S23 to be turned on, so the second driver circuit loop CL2 is turned on in response to the diode S23 within the second switch with freewheeling diode S2, and the direction of the current loop that is turned on of the second driver circuit loop CL2 is counterclockwise, as shown in FIG. 12C. Moreover, if the second energy storage capacitor C2 is not at the voltage saturated status within the time interval T2, then the second energy storage capacitor C2 will be charged during the discharging of the inductor L.

After the time interval T2 ends (i.e., the inductor L is completely discharged), the second pulse signal generated by the second pulse generator G2 controls the controllable switch S21 within the second switch with freewheeling diode S2 to be turned on within a time interval T3, the second energy storage capacitor C2 is equivalent to a voltage source and starts to discharge so that the second driver circuit loop CL2 is turned on, the direction of the current loop after turned on is clockwise, and the current value of the inductor L increases gradually within the time interval T3, as shown in FIG. 12A and FIG. 12D. It shall be appreciated that, the “increase” and “decrease” of the current value herein refer to the “increase” and “decrease” of the absolute values of the current values of the inductor L, and do not represent the flowing direction of the current. Therefore, as shall be appreciated by those of ordinary skill in the art, the positive and negative values of the current in FIG. 12A represent directions of the current.

When the time interval T3 ends, the second pulse signal generated by the second pulse generator G2 controls the controllable switch S21 within the second switch with freewheeling diode S2 to be turned off, and the second driver circuit loop CL2 is turned off instantly, similar to the time interval T2. In response to this, the inductor L serves as a current source in the driver circuit within a time interval T4 and starts to discharge and the current value thereof decreases gradually within the time interval T4. Within the time interval T4, the discharge voltage of the inductor L enables the diode S13 to be turned on, so the first driver circuit loop CL1 is turned on in response to the diode S13 within the first switch with freewheeling diode S1, and the direction of the current loop that is turned on of the first driver circuit loop CL1 is counterclockwise, as shown in FIG. 12E. If the first energy storage capacitor C1 is not at the voltage saturated status when the inductor L is discharging, then the first energy storage capacitor C1 will be charged during the discharging of the inductor L. T1 to T4 is one complete charge-and-discharge period, and one period consumes time about 250 μs, and there may be 5 to 20 periods within one second, each of which is 250 μs and distributed uniformly between one second. For a bidirectional waveform of 250 μs, an electric field of 84 V/M is required at a depth of 25 mm, so a maximum current peak can be determined according to the value.

It shall be appreciated that, the aforesaid embodiments are all described as an extension of the circuit of FIG. 12A. However, the operation of the circuit shown in FIG. 12B shall be appreciated by those of ordinary skill in the art based on the above description. For example, the operations in FIG. 23 are similar to T1 to T4. The operations of FIG. 20 and FIG. 29 are similar to T1 to T2. If the operations of FIG. 17B and FIG. 26 control the pulse generators G1 and G2, then the two pulse signals will make it being similar to T1 to T4; and if the operations of FIG. 17B and FIG. 26 only control the pulse generator G1, then a single pulse signal will make it being similar to T1 to T2. In FIG. 17B and FIG. 20, because there are only a single bulk capacitor and a single stimulator, only a symmetrical waveform where T1=T2, or T3=T4 can be generated, which is different from the case of FIG. 12A. The above circuit operation may be regarded as a discontinuous conduction mode (DCM) in a power electronic converter.

In other embodiments, the first energy storage capacitor C1 may be coupled to a first voltage booster B1 to charge the first energy storage capacitor C1 when the first driver circuit loop CL1 is at the conducting or non-conducting period (e.g., after the time interval T4, before the next time interval T1), and the second energy storage capacitor C2 may be coupled to a second voltage booster B2 to charge the second energy storage capacitor C2 when the second driver circuit loop CL2 is at the conducting or non-conducting period (e.g., after the time interval T2, before the time interval T3, i.e., at the time interval T5), as shown in FIG. 13. Additionally, in other embodiments, the first energy storage capacitor C1 may be set to be coupled to the first voltage booster B1, and the second energy storage capacitor C2 is not coupled to any voltage booster; or the first energy storage capacitor C1 is not coupled to any voltage booster, and the second energy storage capacitor C2 is coupled to the second voltage booster B2. The first voltage booster B1 and the second voltage booster B2 may be connected to a power supply module (not shown), and the power supply module may be connected to an internal battery of the portable rTMS apparatus, or connected to an external power supply (e.g., a wall outlet or a mobile power supply) via an interface.

Moreover, in other embodiments, the driver circuit DRC further comprises at least one snubber (e.g., a passive snubber or a semi-active snubber). For example, as shown in FIG. 14, the driver circuit DRC comprises a passive snubber SB bridging two sides of the inductor L. In FIG. 15A, the passive snubber SB is constituted by a single capacitor Csn. In FIG. 15B, the passive snubber SB is constituted by a capacitor Csn and a resistor Rsn that are connected in series. In FIG. 15C, the passive snubber SB is constituted by connecting a diode Dsn and a resistor Rsn in parallel which are then connected with the capacitor Csn in series. The passive snubber SB is defined as any of the structures of FIG. 15A to FIG. 15C, and the passive snubbers SB mentioned in the subsequent embodiments are all defined in the same way.

For example, the driver circuit DRC may comprise two passive snubbers SB1 that are respectively connected in parallel with the first switch with freewheeling diode S1 and the second switch with freewheeling diode S2, as shown in FIG. 16A to FIG. 16C. The passive snubbers SB and SB1 can be used to absorb the surge in the driver circuit DRC, thereby preventing the driver circuit DRC from being damaged due to the surge.

Moreover, in the driver circuit DRC, if a first auxiliary switch with freewheeling diode S3 is added, or a second auxiliary switch with freewheeling diode S4 is added, or both the first auxiliary switch with freewheeling diode S3 and the second auxiliary switch with freewheeling diode S4 are added, then as shown in FIG. 17A, the circuit operation will not be influenced according to the aforesaid operational principle. The difference lies in that, if both the first auxiliary switch with freewheeling diode S3 and the second auxiliary switch with freewheeling diode S4 are added, then the common ground type of the two energy storage capacitors can be changed. In other words, the first energy storage capacitor and the second energy storage capacitor originally can only have the positive voltage connected to the negative voltage, and after adopting the four switches, the positive ends may be connected together or the negative ends may be connected together.

A third embodiment of the present invention is as shown in FIG. 17B, which is an extension of FIG. 17A in the second embodiment. In FIG. 17B, the first driver circuit loop CL1 is formed by the inductor L, the energy storage capacitor C, the first switch with freewheeling diode S1 and the first auxiliary switch with freewheeling diode S3 that are connected in series, and the second driver circuit loop CL2 is formed by the inductor L, the energy storage capacitor C, the second switch with freewheeling diode S2 and the second auxiliary switch with freewheeling diode S4 that are connected in series. The aforesaid connection mode is defined as “a connection mode of a full-bridge primary side”.

The first driver circuit loop CL1 comprises the first pulse generator G1 that is coupled to the first switch with freewheeling diode S1 and the first auxiliary switch with freewheeling diode S3 and generating a first pulse signal to control the first switch with freewheeling diode S1 and the first auxiliary switch with freewheeling diode S3. The second driver circuit loop CL2 comprises the second pulse generator G2 coupled to the second switch with freewheeling diode S2 and the second auxiliary switch with freewheeling diode S4 and generating a second pulse signal to control the second switch with freewheeling diode S2 and the second auxiliary switch with freewheeling diode S4. The “auxiliary switch with freewheeling diode” is defined as: another switch with freewheeling diode at a position opposite to the switch with freewheeling diode in respect to the inductor in a driver loop and the energy storage capacitor in the driver loop. The control signal received by the auxiliary switch with freewheeling diode will be the same as the control signal received by the switch with freewheeling diode.

Similarly, the portable rTMS apparatus 1 may further comprise a controller to control the first pulse generator G1 and the second pulse generator G2. The first pulse signal and the second pulse signal enable the first driver circuit loop and the second driver circuit loop to conduct alternately. The first driver circuit loop CL1 and the second driver circuit loop CL2 have magnetic flux driving directions opposite to each other in respect to the inductor L.

Similarly, the portable rTMS apparatus 1 may also further comprise a voltage booster (not shown) that is coupled to the capacitor C to charge the capacitor C when the first driver circuit loop CL1 and the second driver circuit loop CL2 are all at a non-conducting period. Moreover, the voltage booster may be connected to a power supply module (not shown), and the power supply module may be connected to an internal battery of the portable rTMS apparatus, or connected to an external power supply (e.g., a wall outlet or a mobile power supply) via an interface.

In other embodiments, the driver circuit DRC may further comprise at least one passive snubber. For example, referring to FIG. 18, the driver circuit DRC comprises the passive snubber SB bridging two sides of the inductor L.

As another example, referring to FIG. 19, the driver circuit DRC comprises four passive snubbers SB1, and the passive snubbers SB1 are respectively connected with the first switch with freewheeling diode S1, the second switch with freewheeling diode S2, the first auxiliary switch with freewheeling diode S3 and the second auxiliary switch with freewheeling diode S4 in parallel. The snubbers SB1 can be used to absorb the surge in the driver circuit DRC, thereby preventing the driver circuit DRC from being damaged due to the surge.

A fourth embodiment of the present invention is as shown in FIG. 20, which is an extension of the third embodiment. FIG. 20 is a schematic view depicting a circuit of the portable rTMS apparatuses 1 within the present invention. The driver circuit DRC comprises an energy storage capacitor C, a switch TR1, an auxiliary switch TR2, a first freewheeling diode D1, a second freewheeling diode D2, and a pulse generator G. The aforesaid connection mode is defined as “a connection mode of a primary side of two switch converters”.

The pulse generator G is coupled to the switch TR1 and the auxiliary switch TR2 and generating a pulse signal to simultaneously control the switch TR1 and the auxiliary switch TR2. The inductor L, the energy storage capacitor C, the switch TR1 and the auxiliary switch TR2 are connected in series to form a driver circuit loop, and the inductor L, the first freewheeling diode Dl and the second freewheeling diode D2 are connected in series to form a re-charging loop. The “switch” is defined as: an electrical network that is equivalent to a controllable switch on scale of circuit in a household power suppliers. The switch may turn on forward current when a control signal is activated, and it may block the forward voltage when the control signal is deactivated. The term of “freewheeling diode” is explained in the following way: when the driver loop is turned off, the diode of the charging loop is immediately turned on, and thus it is called a freewheeling diode. The “auxiliary switch” is defined as: another switch at a position opposite to the switch in respect to the inductor in a driver loop and the energy storage capacitor in the loop. The control signal received by the auxiliary switch will be the same as that of the switch. The “diode” is defined as: a passive component that can block the reverse voltage and turn on the forward current, or an electrical network that is equivalent to the aforesaid component during the operation of the power supplier circuit. The synchronous rectifier is a technology that can replace the diode and reduce the power consumption, and the principle thereof is to appropriately control the active switch (e.g., MOSFET, BJT or the like) to adjust the time periods where the voltage is blocked and the current is turned on. Therefore, it is equivalent to a diode during the operation of the circuit. Therefore, the synchronous rectifier can be regarded as a diode.

Similarly, the portable rTMS apparatus 1 may further comprise a voltage booster (not shown) that is coupled to the capacitor C to charge the energy storage capacitor C when the driver circuit loop is at a non-conducting period. Moreover, as described in the aforesaid embodiments, the voltage booster may be connected to a power supply module (not shown), and the power supply module (not shown) may be connected to an internal battery of the portable rTMS apparatus, or connected to an external power supply (e.g., a wall outlet or a mobile power supply) via an interface.

In other embodiments, the driver circuit DRC further comprises at least one passive snubber. For example, referring to FIG. 21, the driver circuit DRC comprises the passive snubber SB bridging two sides of the inductor L. As another example, in other embodiments, referring to FIG. 22, the driver circuit DRC comprises two passive snubbers SB1 that respectively connected in parallel with the switch TR1 and the auxiliary switch TR2.

A fifth embodiment of the present invention is as shown in FIG. 23, which is an extension of the second embodiment. FIG. 23 is a schematic view depicting a circuit of the portable rTMS apparatuses 1 within the present invention. The driver circuit DRC comprises a first energy storage capacitor C1, a second energy storage capacitor C2, a first switch with freewheeling diode S1, a second switch with freewheeling diode S2, a first pulse generator G1 and a second pulse generator G2 which are presented in the form similar to a flyback converter. Similarly, the first energy storage capacitor C1, the first switch with freewheeling diode S1 and a primary coil of the inductor L are connected in series to form the first driver circuit loop CL1, and the second energy storage capacitor C2, the second switch with freewheeling diode S2, and the secondary coil of the inductor L are connected in series to form the second driver circuit loop CL2. It shall be appreciated that, the schematic view of the circuit in FIG. 25 is not intended to limit the sequence of the first energy storage capacitor C1 and the first switch with freewheeling diode S1 in the first driver circuit loop CL1 and the sequence of the second energy storage capacitor C2 and the second switch with freewheeling diode S2 in the second driver circuit loop CL2. In practical application, the provider or the manufacturer of the portable rTMS apparatus 1 may decide the order in which the capacitors and the switches are connected in series according to different requirements. The aforesaid connection mode is defined as “a connection mode of a bidirectional flyback converter”.

The primary coil of the inductor L and the secondary coil of the inductor L being the two coils of a stimulator. The first pulse signal and the second pulse signal enable the first driver circuit loop CL1 and the second driver circuit loop CL2 to be conduct alternately. The first driver circuit loop CL1 and the second driver circuit loop CL2 have magnetic flux driving directions opposite to each other in respect to the inductor L.

Similarly, the first energy storage capacitor C1 may be coupled to a voltage booster (not shown) to charge the first energy storage capacitor C1 when the first driver circuit loop CL1 is at a non-conducting period, and the second energy storage capacitor C2 may also be coupled to a voltage booster (not shown) to charge the second energy storage capacitor C2 when the second driver circuit loop CL2 is at a non-conducting period. Moreover, in other embodiments, the voltage booster coupled to the first energy storage capacitor C1 and the voltage booster coupled to the second energy storage capacitor C2 may be connected to a power supply module (not shown), and the power supply module may be connected to an internal battery of the portable rTMS apparatus, or connected to an external power supply (e.g., a wall outlet or a mobile power supply) via an interface.

Moreover, in other embodiments, the driver circuit DRC further comprises at least one passive snubber connected to the first switch with freewheeling diode S1 and the second switch with freewheeling diode S2. For example, as shown in FIG. 24, two passive snubbers SB1 are respectively connected in parallel with the first switch with freewheeling diode S1 and the second switch with freewheeling diode S2.

Moreover, in other embodiments, the driver circuit DRC may comprise an energy-recovering snubber SB3 as shown in FIG. 25. The “energy-recovering snubber” is defined as a snubber constituted by diodes Dsn1 and Dsn2, an inductor Lsn and a capacitor Csn. In the first driver circuit loop CL1, the diodes Dsn1 and Dsn2, the inductor Lsn and the energy storage capacitor C1 are connected in series to form a loop, and the diodes Dsn1 and Dsn2 are connected to an endpoint between the inductor L and the first switch with freewheeling diode S1 via a capacitor Csn therebetween. The second driver circuit loop CL2 is connected in the same way. The energy-recovering snubbers SB3 mentioned in the subsequent embodiments are all defined in the same way. Additionally, in other embodiments, a coupling capacitor may bridge two sides of the inductor L, and when the coupling capacitance is very small, the function of the coupling capacitor in the driver circuit DRC is equivalent to a snubber that is configured to filter the surge in the circuit. The first energy storage capacitor C1 and the second energy storage capacitor C2 may be common grounded in various manners, e.g., connecting positive ends together, connecting positive ends and negative ends together or the like, all of which belong to reasonable circuit types. It shall be appreciated that, the inductor Lsn refers to an inductor that is specially added other than the stimulator, and the inductor L in other figures all refers to the stimulator.

A sixth embodiment of the present invention is as shown in FIG. 26. FIG. 26 is a schematic view depicting a circuit of the portable rTMS apparatuses 1 within the present invention. The driver circuit DRC comprises an energy storage capacitor C, a first switch with freewheeling diode S1, a second switch with freewheeling diode S2, a first pulse generator G1 and a second pulse generator G2 which are presented in the form similar to a push-pull converter. The aforesaid connection mode is defined as “a connection mode of a primary side of a push-pull converter”. FIG. 26 is represented by a center-tapped inductor, which can also be equivalent to two coupling inductors common grounded, and then the two switches with freewheeling diode may be modified to be located at opposite sides of the capacitor.

In this embodiment, the first driver circuit loop CL1 is formed by connecting the primary coil of the inductor L, the energy storage capacitor C and the first switch with freewheeling diode S1 in series, and the second driver circuit loop CL2 is formed by connecting the secondary coil of the inductor L, the energy storage capacitor C and the second switch with freewheeling diode S2 in series. Similarly, the first pulse signal generated by the first pulse generator G1 and the second pulse signal generated by the second pulse generator G2 enable the first driver circuit loop CL1 and the second driver circuit loop CL2 to conduct alternately, and the first driver circuit loop CL1 and the second driver circuit loop CL2 have magnetic flux driving directions opposite to each other in respect to the inductor. Similarly, in other embodiments, the portable rTMS apparatus 1 of the present invention may further comprise a controller for controlling the first pulse generator G1 and the second pulse generator G2.

Similarly, the portable rTMS apparatus may further comprise a voltage booster (not shown) that is coupled to the capacitor C to charge the energy storage capacitor C when the first driver circuit loop and the second driver circuit loop are all at a non-conducting period. The voltage booster may be connected to a power supply module (not shown), and the power supply module may be connected to an internal battery of the portable rTMS apparatus, or connected to an external power supply (e.g., a wall outlet or a mobile power supply) via an interface.

In other embodiments, the driver circuit DRC further comprises at least one passive snubber connected to the first switch with freewheeling diode S1 and the second switch with freewheeling diode S2. For example, as shown in FIG. 27, two passive snubbers SB1 are respectively connected with the first freewheeling diode switch S1 and the second switch with freewheeling diode S2 in parallel. Moreover, in other embodiments, the driver circuit DRC of this embodiment may also adopt the energy-recovering snubber SB3 of the aforesaid embodiments, as shown in FIG. 28.

A seventh embodiment of the present invention is as shown in FIG. 29. FIG. 29 is a schematic view depicting a circuit of the portable rTMS apparatuses 1 within the present invention. The driver circuit DRC comprises an energy storage capacitor C, a switch TR, a freewheeling diode D, and a pulse generator G which are presented in the form similar to a push-pull converter. The pulse generator G is coupled to the switch TR and generating a pulse signal to control the switch TR. When the pulse generator G controls to turn on the switch TR, the inductor L, the energy storage capacitor C and the switch TR connected in series form a driver circuit loop. When the pulse generator G controls to turn off the switch TR, the inductor L, the energy storage capacitor C and the freewheeling diode D connected in series form a re-charging loop. The aforesaid connection mode is defined as “a connection mode of the primary side of a simplified push-pull converter”. FIG. 29 is represented by a center-tapped inductor, which may also be equivalent to two coupling inductors common grounded, and then the freewheeling diode switch and the diode may be modified to be located at opposite sides of the capacitor.

Similarly, the portable rTMS apparatus may further comprise a voltage booster (not shown) that is coupled to the energy storage capacitor C to charge the energy storage capacitor C when the driver circuit loop DRC is at a non-conducting period. In other embodiments, the driver circuit DRC further comprises the passive snubber SB1 connected to the switch TR, as shown in FIG. 30. Moreover, in other embodiments, the driver circuit DRC of this embodiment may also adopt the energy-recovering snubber SB3 of the aforesaid embodiments, as shown in FIG. 31.

An eighth embodiment of the present invention is as shown in FIG. 32. FIG. 32 is a schematic view depicting a circuit of the portable rTMS apparatuses 1 within the present invention. The driver circuit DRC comprises a first energy storage capacitor C1, a second energy storage capacitor C2, a first switch with freewheeling diode S1, a second switch with freewheeling diode S2, a first pulse generator G1 and a second pulse generator G2. In this embodiment, the first driver circuit loop CL1 is formed by connecting the inductor L, the first energy storage capacitor C1, the second energy storage capacitor C2 and the first switch with freewheeling diode S1 in series, and the second driver circuit loop CL2 is formed by connecting the inductor L, the second energy storage capacitor C2 and the second switch with freewheeling diode S2 in series. The aforesaid connection mode is defined as “a connection mode of a bidirectional battery charger”.

The first pulse generator G1 is coupled to the first switch with freewheeling diode S1 and generating a first pulse signal to control the first switch with freewheeling diode 1. The second pulse generator G2 is coupled to the second switch with freewheeling diode S2 and generating a second pulse signal to control the second switch with freewheeling diode S2. Similarly, in other embodiments, the portable rTMS apparatus 1 of the present invention may further comprise a controller for controlling the first pulse generator G1 and the second pulse generator G2. The first pulse signal and the second pulse signal enable the first driver circuit loop CL1 and the second driver circuit loop CL2 to conduct alternately. The first driver circuit loop CL1 and the second driver circuit loop CL2 have magnetic flux driving directions opposite to each other. The operation mode of this embodiment is the same as the bidirectional battery charger of the well-known technology, and the principle thereof is different from the second to the seventh embodiments.

Like the aforesaid embodiments, in the driver circuit DRC of this embodiment, the first energy storage capacitor C1 may be coupled to a voltage booster (not shown) to charge the first capacitor C1 when the first driver circuit loop is at a non-conducting period, and the second energy storage capacitor C2 may be coupled to another voltage booster (not shown) to charge the second capacitor C2 when the second driver circuit loop is at a non-conducting period. Moreover, in other embodiments, the voltage booster may be connected to a power supply module (not shown), and the power supply module may be connected to an internal battery of the portable rTMS apparatus, or connected to an external power supply (e.g., a wall outlet or a mobile power supply) via an interface. Additionally, in other embodiments, the driver circuit DRC further comprises at least one passive snubber. For example, referring to FIG. 33, the driver circuit DRC comprises the passive snubber SB bridging two sides of the inductor L. As another example, in other embodiments, referring to FIG. 34, the driver circuit DRC comprises two passive snubbers SB1 that are respectively connected in parallel with the first switch with freewheeling diode S1 and the second switch with freewheeling diode S2.

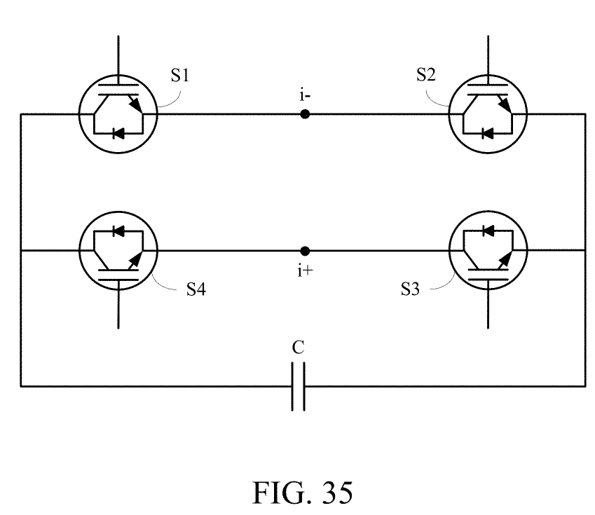

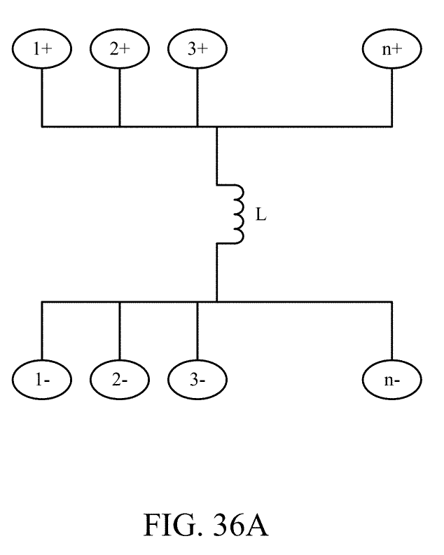

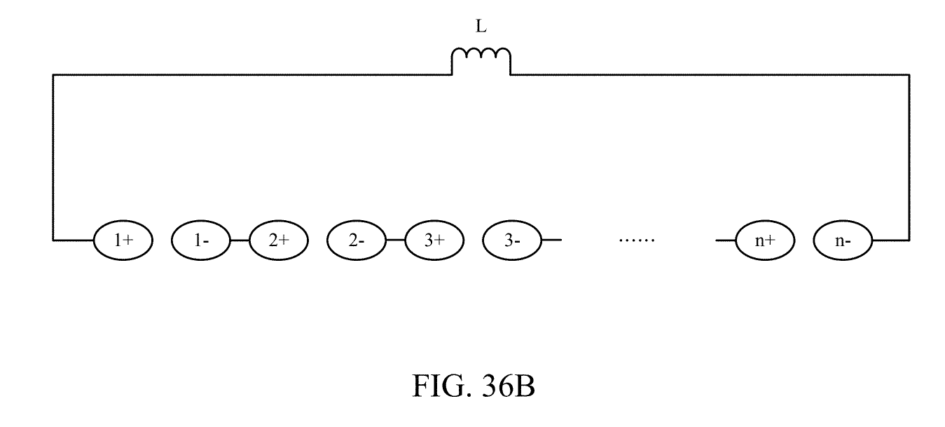

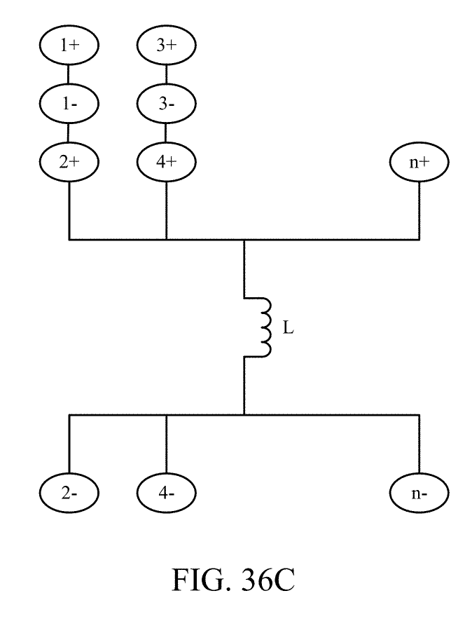

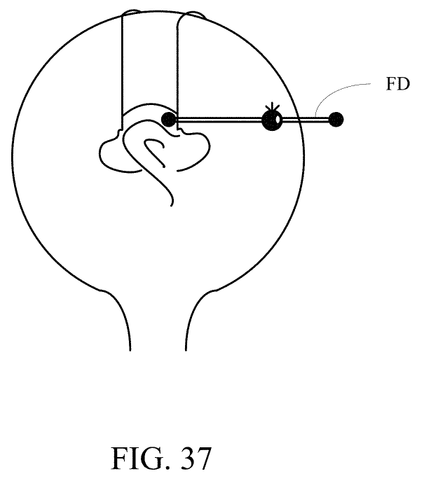

A ninth embodiment of the present invention is as shown in FIG. 35, FIG. 36A to FIG. 36C. The ninth embodiment is an extension of the second embodiment to the eighth embodiment. FIG. 35 depicts a schematic view of an i^{th} driver circuit of a portable rTMS apparatuses 1 according to an embodiment of the present invention. To simplify the description, elements unrelated to this embodiment have been omitted and not depicted, and other components required during the operation of the driver circuit shall be appreciated by those of ordinary skill in the art based on the aforesaid embodiments and thus will not be further described herein. In this embodiment, two ends of the inductor L in the driver circuit (not shown) are distinguished respectively as a positive end and a negative end, i+ represents the positive connection point of the i^{th} driver circuit, i− represents the negative connection point of the i^{th} driver circuit. If the voltage or current of a single driver circuit is insufficient, then a plurality of driving loops may be superimposed to achieve the driving effect.

For example, in the case where the voltage of a single driving loop is sufficient to drive the inductor L but the current is insufficient, circuit configuration of FIG. 36A may be used, in which n driver circuits are connected in parallel to obtain sufficient current, the positive connection point of each driver circuit is connected to the positive end of the inductor L, and the negative connection point of each driver circuit is connected to the negative end of the inductor L.

As another example, in the case where the current of a single driving loop is sufficient to drive the inductor L but the voltage is insufficient, circuit configuration of FIG. 36B may be used, in which a plurality of driver circuits are connected in series to obtain sufficient voltage. In the driver circuit of FIG. 36B, only a positive connection point 1+ of the first driver circuit is connected to the positive end of the inductor L, and a negative connection point n− of the n^{th} driver circuit is connected to the negative end of the inductor L. When using the series connection configuration, the passive snubber should still be connected at two sides of the stimulator inductor, and the capacitor Csn and the diode Dsn1 of the energy-recovering snubber still bridge two sides of the inductor, and the diode Dsn2 and the inductor Lsn are connected to the sub-circuit where the capacitor Csn is located. If the energy storage capacitor uses the superimposed structure, the sum of the energy storage of the energy storage capacitors of all the sub-circuits should be greater than 2.5 J.

In other embodiments, a plurality of driver circuits may also be partially connected in series and partially connected in parallel to obtain enough current and voltage, as shown in FIG. 36C, and the circuit principle thereof is deducted according to the superposition characteristics of a lumped element circuit in the linear system. What described above is the principle of a cascode converter.

It shall be appreciated that, this embodiment takes the driver circuit of the third embodiment for illustration, and the operation of the series connection and parallel connection of the driver circuit of other embodiments shall be appreciated by those of ordinary skill in the art based on the above description, and thus will not be further described herein. In other words, in this embodiment, in addition to the driver circuit of the aforesaid embodiments, a plurality of other driver circuits may be further included, and each of the other driver circuits will be the same as the original driver circuit. Based on different design requirements, these other driver circuits may be connected with the original driver circuit in parallel and then connected to the inductor L, or these other driver circuits may be connected with the original driver circuit in series and then connected to the inductor L, or these other driver circuits may even have a part thereof connected with the original driver circuit in parallel and another part thereof connected with the original driver circuit in series and then connected to the inductor L.

A tenth embodiment of the present invention is as shown in FIG. 44. The housing carrying the inductor L may be designed to have an appearance similar to an earphone so as to be conveniently fastened on the head. However, in order to fix the position of the stimulator (i.e., the inductor L), this new model further provides a fastening device FD connected to the housing carrying the inductor L, and uses end points of the auricle, the eyes and the fastening device as the positioning points, thereby preventing the position of the stimulator from being shifted during the use thereof. Additionally, in other embodiments, a plurality of stimulators may be disposed by adopting a plurality of inductors L that are connected in parallel, and this shall be accomplished by those of ordinary skill in the art based on the aforesaid embodiments, and thus will not be further described herein. With the development of materials in the future, it is more possible to integrate the driver circuit and the stimulator on the head device.

According to the above descriptions, the portable rTMS apparatus of the present invention uses a specially designed stimulator (inductor) and circuit to reduce the power consumption required during the use thereof, improve the heat-dissipating capability and reduce the overall volume of the apparatus, thereby achieving the function of being portable. Accordingly, as compared to the conventional rTMS apparatus, the portable rTMS apparatus of the present invention is not limited by the environment when using, and can be powered by a built-in battery or a mobile power supply.

It shall be appreciated that, these embodiments of the present invention are only for disclosure of implementation contents and technical feature, not for limiting the present invention coverage. Besides, people skilled in this field may proceed with a variety of modifications and replacements based on the disclosures and suggestions without departing from the characteristics, they have substantially been covered in the following claims as appended, and the scope claimed in this application shall be governed by the claims

### BRIEF DESCRIPTION OF REFERENCE NUMERALS

1: portable repetitive transcranial magnetic stimulation apparatus
L: inductor
C1: first energy storage capacitor
C2: second energy storage capacitor
S1: first switch with freewheeling diode
S11: controllable switch
S13: diode
S2: second switch with freewheeling diode
S21: controllable switch
S23: diode
S3: first auxiliary switch with freewheeling diode
S4: second auxiliary switch with freewheeling diode
TR: switch
TR1: first switch
TR2: second switch
D: freewheeling diode
D1: first freewheeling diode
D2: second freewheeling diode
G: pulse generator
G1: first pulse generator
G2: second pulse generator
CL1: first driver circuit loop
CL2: second driver circuit loop
T1-T5: time interval
B1: first voltage booster
B2: second voltage booster
SB: passive snubber
SB1: passive snubber
SB3: energy-recovering snubber
COE: core
COL: coil
UCOL: upper part
LCOL: lower part
INT: connecting portion
UFC: upper fastening component
LFC: lower fastening component
DRC: driver circuit
CG: groove
BR1: upper blocking component
MS: magnetostrictive material
BR2: lower blocking component
SD: stimulating site
H: housing
CS: ceramic substrate
SCS: extended ceramic substrate
MP: middle part
EP1: first end
EP2: second end
C: energy storage capacitor
Csn: capacitor
Rsn: resistor
Dsn, Dsn1, Dsn2: diode
Lsn: inductor
i+: positive connection point of the i^{th} driver circuit
i−: negative connection point of the i^{th} driver circuit
1+: positive connection point of the first driver circuit
1−: negative connection point of the first driver circuit
2+: positive connection point of the second driver circuit
2−: negative connection point of the second driver circuit
3+: positive connection point of the third driver circuit
3−: negative connection point of the third driver circuit
4+: positive connection point of the fourth driver circuit
4−: negative connection point of the fourth driver circuit
n+: positive connection point of the n^{th} driver circuit
n−: negative connection point of the n^{th} driver circuit
FD: fastening device.

1. A portable repetitive transcranial magnetic stimulation (rTMS) apparatus, comprising:
a driver circuit; and
an inductor, being used as a stimulator, connected to the driver circuit;
wherein the inductor is composed of a core and at least one coil, the core has a groove, the at least one coil has an upper part and a lower part, the upper part of the at least one coil is configured to be distant to the core and pass through an upper side, or a right side, or a left side of the core, and the lower part of the at least one coil is configured to pass through the groove of the core.

2. The portable rTMS apparatus of claim 1, wherein the upper part of the at least one coil is distant to the core, the groove has a width between 0.7 cm and 11.2 cm, the groove and an average current of the lower part of the at least one coil have a bending angle of less than 60°, and the core has a length between 0.7 cm and 11.2 cm and a thickness between 0.4 cm and 4 cm.

3. The portable rTMS apparatus of claim 1, further comprising an upper fastening component and a lower fastening component, wherein the upper part of the at least one coil is further configured to be fastened by the upper fastening component, the lower part of the at least one coil is further configured to be fastened by the lower fastening component, the lower fastening component includes a plurality of ceramic substrates, each of the ceramic substrates has a middle part, a first end and a second end, the first end and the second end are connected by the middle part, and the ceramic substrates are stacked into a layered structure.

4. The portable rTMS apparatus of claim 3, wherein for any two adjacent ceramic substrates among the ceramic substrates, a size of an upper ceramic substrate is smaller than that of a lower ceramic substrate so that a ladder shape is formed by the first ends and the second ends.

5. The portable rTMS apparatus of claim 2, wherein the upper part of the at least one coil is more than 7 mm away from the core, the width of the groove is between 1.4 cm and 5.6 cm, the length of the core is between 1.4 cm and 5.6 cm, and the thickness of the core is between 0.7 cm and 2.8 cm.

6. The portable rTMS apparatus of claim 2, wherein the groove has a depth between 0.7 cm and 4 cm.

7. The portable rTMS apparatus of claim 2, wherein the core is composed of a plurality of iron core sheets arranged in a direction perpendicular to a current direction, the core is formed of a high saturation flux density material with a saturation density greater than 1.3 T, and each of the iron core sheets has a thickness less than 1 mm.

8. The portable rTMS apparatus of claim 2, wherein the core is composed of a plurality of iron core sheets arranged in a direction perpendicular to a current direction, the core is formed of a high saturation flux density material with a saturation density greater than 1.9 T, and each of the iron core sheets has a thickness less than 0.5 mm.

9. The portable rTMS apparatus of claim 1, wherein the inductor further includes a plurality of extended ceramic substrates that contact with the ceramic substrates respectively along two side portions of the core or are integrated with the ceramic substrates.

10. The portable rTMS apparatus of claim 1, wherein the inductor further includes an upper blocking component made of a high hardness material, and the core of the inductor is at least partly covered by the upper blocking component.

11. The portable rTMS apparatus of claim 1, wherein the inductor further includes a lower blocking component made of a high hardness material, and two side portions of the core of the inductor are connected by the lower blocking component.

12. The portable rTMS apparatus of claim 11, wherein the lower blocking component further includes a magnetostrictive material.

13. The portable rTMS apparatus of claim 1, further comprising a housing with high acoustic impedance, wherein the core is disposed inside the housing, the housing and the core are separated by at least a layer of substance having low acoustic impedance therebetween, the layer of substance having low acoustic impedance has a thickness ranging between 0.4 cm and 8 cm, the housing and the core are connected by a spring having a coefficient between 0.001 mm/N and 0.1 mm/N, and the housing has a thickness ranging between 0.5 mm and 1 cm.

14. The portable rTMS apparatus of claim 2, wherein the driver circuit comprises:
a first energy storage capacitor;
a second energy storage capacitor;
a first switch with freewheeling diode;
a second switch with freewheeling diode;
a first pulse generator coupled to the first switch with freewheeling diode, generating a first pulse signal to control the first switch with freewheeling diode; and
a second pulse generator coupled to the second switch with freewheeling diode, generating a second pulse signal to control the second switch with freewheeling diode;
wherein the inductor, the first energy storage capacitor and the first switch with freewheeling diode are connected in series to form a first driver loop, the inductor, the second energy storage capacitor and the second switch with freewheeling diode are connected in series to form a second driver loop, the first pulse signal and the second pulse signal enable the first driver loop and the second driver loop to conduct alternately, and the first driver loop and the second driver loop have magnetic flux driving directions opposite to each other in respect to the inductor.

15. The portable rTMS apparatus of claim 2, wherein the driver circuit comprises:
a first energy storage capacitor;
a second energy storage capacitor;
a first switch with freewheeling diode;
a second switch with freewheeling diode;
a first pulse generator coupled to the first switch with freewheeling diode, generating a first pulse signal to control the first switch with freewheeling diode; and
a second pulse generator coupled to the second switch with freewheeling diode, generating a second pulse signal to control the second switch with freewheeling diode;
wherein the inductor, the first energy storage capacitor, the second energy storage capacitor and the first switch with freewheeling diode are connected in series to form a first driver loop, the inductor, the second energy storage capacitor and the second switch with freewheeling diode are connected in series to form a second driver loop, the first pulse signal and the second pulse signal enable the first driver loop and the second driver loop to conduct alternately, and the first driver loop and the second driver loop have magnetic flux driving directions opposite to each other in respect to the inductor.

16. The portable rTMS apparatus of claim 14, wherein the driver circuit further comprises at least one passive snubber connected to the first switch with freewheeling diode and the second switch with freewheeling diode or connected to the inductor.

17. The portable rTMS apparatus of claim 2, wherein the driver circuit comprises:
an energy storage capacitor;
a first switch with freewheeling diode;
a first auxiliary switch with freewheeling diode;
a second switch with freewheeling diode;
a second auxiliary switch with freewheeling diode;
a first pulse generator coupled to the first switch with freewheeling diode and the first auxiliary switch with freewheeling diode, generating a first pulse signal to control the first switch with freewheeling diode and the first auxiliary switch with freewheeling diode; and
a second pulse generator coupled to the second switch with freewheeling diode and the second auxiliary switch with freewheeling diode, generating a second pulse signal to control the second switch with freewheeling diode and the second auxiliary switch with freewheeling diode;
wherein the inductor, the energy storage capacitor, the first switch with freewheeling diode and the first auxiliary switch with freewheeling diode are connected in series to form a first driver loop, the inductor, the energy storage capacitor, the second switch with freewheeling diode and the second auxiliary switch with freewheeling diode are connected in series to form a second driver loop, and the first driver loop and the second driver loop have magnetic flux driving directions opposite to each other in respect to the inductor.

18. The portable rTMS apparatus of claim 17, wherein the driver circuit further comprises at least one passive snubber connected to the first switch with freewheeling diode, the first auxiliary switch with freewheeling diode, the second switch with freewheeling diode and the second auxiliary switch with freewheeling diode, or connected to the inductor.

19. The portable rTMS apparatus of claim 2, wherein the driver circuit comprises:
an energy storage capacitor;
a switch;
an auxiliary switch;
a first freewheeling diode;
a second freewheeling diode; and
a pulse generator coupled to the switch and the auxiliary switch, generating a pulse signal to control the switch and the auxiliary switch;
wherein the inductor, the energy storage capacitor, the switch and the auxiliary switch are connected in series to form a driver loop, and the inductor, the first freewheeling diode and the second freewheeling diode are connected in series to form a charging loop.

20. The portable rTMS apparatus of claim 19, wherein the driver circuit further comprises at least one passive snubber connected to the switch and the auxiliary switch or connected to the inductor.

21. The portable rTMS apparatus of claim 2, wherein the driver circuit comprises:
a first energy storage capacitor;
a second energy storage capacitor;
a first switch with freewheeling diode;
a second switch with freewheeling diode;
a first pulse generator coupled to the first switch with freewheeling diode, generating a first pulse signal to control the first switch with freewheeling diode; and
a second pulse generator coupled to the second switch with freewheeling diode, generating a second pulse signal to control the second switch with freewheeling diode;
wherein the at least one coil includes a primary coil and a secondary coil, the primary coil, the first energy storage capacitor and the first switch with freewheeling diode are connected in series to form a first driver loop, the secondary coil, the second energy storage capacitor and the second switch with freewheeling diode are connected in series to form a second driver loop, the first pulse signal and the second pulse signal enable the first driver loop and the second driver loop to conduct alternately, and the first driver loop and the second driver loop have magnetic flux driving directions opposite to each other in respect to the inductor.

22. The portable rTMS apparatus of claim 2, wherein the driver circuit comprises:
an energy storage capacitor;
a first switch with freewheeling diode;
a second switch with freewheeling diode;
a first pulse generator coupled to the first switch with freewheeling diode, generating a first pulse signal to control the first switch with freewheeling diode; and
a second pulse generator coupled to the second switch with freewheeling diode, generating a second pulse signal to control the second switch with freewheeling diode;
wherein the at least one coil includes a primary coil and a secondary coil, the primary coil, the energy storage capacitor and the first switch with freewheeling diode are connected in series to form a first driver loop, the secondary coil, the energy storage capacitor and the second switch with freewheeling diode are connected in series to form a second driver loop, the first pulse signal and the second pulse signal enable the first driver loop and the second driver loop to conduct alternately, and the first driver loop and the second driver loop have magnetic flux driving directions opposite to each other in respect to the inductor.

23. The portable rTMS apparatus of claim 21, wherein the driver circuit further comprises at least one passive snubber connected to the first switch with freewheeling diode and the second switch with freewheeling diode, or at least one energy-recovering snubber connected to the inductor.

24. The portable rTMS apparatus of claim 2, wherein the driver circuit comprises:
an energy storage capacitor;
a switch;
a freewheeling diode; and
a pulse generator coupled to the switch, generating a pulse signal to control the switch;
wherein the at least one coil includes a primary coil and a secondary coil, the primary coil, the energy storage capacitor and the switch are connected in series to form a driver loop, and the secondary coil, the energy storage capacitor and the freewheeling diode are connected in series to form a charging loop.

25. The portable rTMS apparatus of claim 24, wherein the driver circuit further comprises one passive snubber connected to the switch or at least one energy-recovering snubber connected to the inductor.

26. The portable rTMS apparatus of claim 2, wherein a first number of conductors of the lower part of the coil is smaller than a second number of conductors of the upper part of the coil.

27. The portable rTMS apparatus of claim 2, wherein the upper part of the coil is in the form of a plate-shaped structure.

28. The portable rTMS apparatus of claim 2, wherein a cross-sectional area of conductors of the upper part of the coil is larger than a cross-sectional area of conductors of the lower part of the coil.

29. The portable rTMS apparatus of claim 2, wherein a total conductor spacing of the upper part of the coil is larger than that of the lower part of the coil.

30. The portable rTMS apparatus of claim 2, further comprising an upper fastening component and a lower fastening component, wherein the upper part of the at least one coil is further configured to be fastened by the upper fastening component, the lower part of the at least one coil is further configured to be fastened by the lower fastening component, the lower fastening component includes a plurality of ceramic substrates, each of the ceramic substrates has a middle part, a first end and a second end, the first end and the second end are connected by the middle part, and the ceramic substrates are stacked into a layered structure.

31. The portable rTMS apparatus of claim 30, wherein for any two adjacent ceramic substrates among the ceramic substrates, a size of an upper ceramic substrate is smaller than that of a lower ceramic substrate so that a ladder shape is formed by the first ends and the second ends.

32. The portable rTMS apparatus of claim 5, wherein the groove has a depth between 0.7 cm and 4 cm.

33. The portable rTMS apparatus of claim 15, wherein the driver circuit further comprises at least one passive snubber connected to the first switch with freewheeling diode and the second switch with freewheeling diode or connected to the inductor.

34. The portable rTMS apparatus of claim 22, wherein the driver circuit further comprises at least one passive snubber connected to the first switch with freewheeling diode and the second switch with freewheeling diode, or at least one energy-recovering snubber connected to the inductor.
